(19) United States
(12) Patent Application Publication
(10) Pub. No.: US 2016/0122753 A1
(43) Pub. Date: May 5, 2016

(54) HIGH-THROUGHPUT RNA-SEQ

(71) Applicants: Tarjei MIKKELSEN, Cambridge, MA (US); Magali SOUMILLON, Cambridge, MA (US); PRESIDENT AND FELLOWS OF HARVARD COLLEGE, Cambridge, MA (US); THE BROAD INSTITUTE, INC., Cambrige, MA (US)

(72) Inventors: Tarjei Mikkelsen, Cambridge, MA (US); Magali Soumillon, Cambridge, MA (US)

(21) Appl. No.: 14/898,030

(22) PCT Filed: Jun. 12, 2014
(86) PCT No.: PCT/US14/42159

§ 371 Date: Dec. 11, 2015

Related U.S. Application Data
(60) Provisional application No. 61/834,163, filed on Jun. 12, 2013.

(51) Int. Cl. C12N 15/10

(52) U.S. Cl. CPC C12N 15/1065

(57) ABSTRACT

The present invention relates generally to methods for single-cell nucleic acid profiling, and nucleic acids useful in those methods. For example, it concerns using barcode sequences to track individual nucleic acids at single-cell resolution, utilizing template switching and sequencing reactions to generate the nucleic acid profiles. These methods and compositions are also applicable to other starting materials, such as cell and tissue lysates or extracted/purified RNA.

### RELATED APPLICATION

This application claims priority and benefit from U.S. Provisional Patent Application No. 61/834,163, filed Jun. 12, 2013, the contents and disclosures of which are hereby incorporated by reference in their entirety.

### FIELD OF THE INVENTION

The present invention relates generally to methods for single-cell nucleic acid profiling, and nucleic acids useful in those methods. In some embodiments, it concerns using barcode sequences to track individual nucleic acids at single-cell resolution, utilizing template switching and sequencing reactions to generate the nucleic acid profiles. In addition to the substantial utility in single cell profiling, the methods and compositions provided herein are also applicable to other starting materials, such as cell and tissue lysates or extracted/purified RNA.

### BACKGROUND OF THE INVENTION

Although transcriptome profiling is an important method for functional characterization of cells and tissues, current technical limitations for whole transcriptome analysis limit the technique to either population averages or to a limited number of single cells. These shortcomings limit transcriptome profiling's ability to accurately assess stochastic variation in gene expression between individual cells and the analysis of distinct subpopulations of cells, both of which have been proposed to be important factors driving cellular differentiation and tissue homeostasis. In addition, current single-cell transcriptome profiling methods, in addition to being limited to a relatively low number of cells, also are expensive and labor-intensive. Improved methods are therefore required to fully characterize a cell population at single-cell resolution. Such improved methods also have utility in improving analysis of other starting materials, such as cell and tissue lysates or extracted/purified RNA.

### SUMMARY OF THE INVENTION

In some embodiments, the invention provides a nucleic acid comprising a 5′ poly-isonucleotide sequence (for example, comprising an isocytosine, an isoguanosine, or both, such as an isocytosine-isoguanosine-isocytosine sequence), an internal adapter sequence, and a 3′ guanosine tract. The 3′ guanosine tract can comprise two guanosines, three guanosines, four guanosines, five guanosines, six guanosines, seven guanosines, or eight guanosines. In certain embodiments, the 3′ guanosine tract comprises three guanosines. The adapter sequence can be 12 to 32 nucleotides in length, for example, 22 nucleotides in length (e.g., an adapter sequence of 5′-ACACTCTTTCCCTACACGACGC-3′ (SEQ ID NO: 1)).

In some embodiments, the invention provides a nucleic acid comprising a 5′ blocking group (e.g., biotin or an inverted nucleotide), an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine. In certain embodiments, the internal adapter sequence is 23 to 43 nucleotides in length, for example, 33 nucleotides in length (e.g., an internal adapter sequence of 5′-ACACTCTTTCCCTACACGACGC-3′ (SEQ ID NO: 1)). In certain embodiments, the barcode sequence is 4 to 20 nucleotides in length, for example, 6 nucleotides in length. In certain embodiments, the UMI sequence is six to 20 nucleotides in length, for example, ten nucleotides in length. In some embodiments, the complementarity sequence is a poly(T) sequence, and may be 20 to 40 nucleotides in length, for example, 30 nucleotides in length.

In some embodiments, the invention provides a kit comprising one or more nucleic acids as described above, for example a) a nucleic acid comprising a 5′ poly-isonucleotide sequence, an internal adapter sequence, and a 3′ guanosine tract, b) a nucleic acid comprising a 5′ blocking group (e.g., biotin or an inverted nucleotide), an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine, or c) both. In certain embodiments, the kit comprises a plurality of the nucleic acids of b). In further embodiments, the UMI sequence of each nucleic acid in the plurality of nucleic acids is unique among the nucleic acids in the kit, and in still further embodiments, the plurality of nucleic acids comprises different populations of nucleic acid species. In such embodiments, each population of nucleic acid species may comprise a different barcode sequence that uniquely identifies a single population of nucleic acid species. In certain embodiments, each population of nucleic acid species is in a separate container, and the bar code of each population of nucleic acid species differs by at least two nucleotides from the bar code of each other population of nucleic acid species.

A kit of the invention may further comprise a third nucleic acid primer comprising 12 to 32 nucleotides (e.g., 22 nucleotides in length) and a 5′ blocking group (e.g., biotin or an inverted nucleotide). An exemplary sequence of such a primer is 5′-ACACTCTTTCCCTACACGACGC-3′ (SEQ ID NO: 2). A kit may further comprise a nucleic acid comprising a barcode sequence, and optionally also comprise a phosphorothioate bond-containing nucleic acid comprising an X1*X2*X3*X4*X5*3′ sequence, wherein * is a phosphorothioate bond. In certain embodiments, the phosphorothioate bond-containing nucleic acid is 48 to 68 nucleotides in length, for example, 58 nucleotides in length. An exemplary sequence of a phosphorothioate bond-containing nucleic acid is AATGATACGGCGACCACCGAGATCTACACTCTTTCCCTACACGACGCTC TTCCG*A*T*C*T*3′ (SEQ ID NO: 3).

In some embodiments, the kit further comprises a capture plate and/or a reverse transcriptase enzyme, such as a Moloney Murine Leukemia Virus (MMLV) reverse transcriptase (e.g., SMARTscribe™ reverse transcriptase or SuperScript II™ reverse transcriptase or Maxima H Minus™ reverse transcriptase) and/or a DNA purification column, such as a DNA purification spin column, and/or a protease or proteinase (e.g., proteinase K).

In some embodiments, the invention provides a method for gene profiling, comprising a) providing a plurality of single cells; b) releasing mRNA from each single cell to provide a plurality of individual mRNA samples, wherein each individual mRNA sample is from a single cell; c) reverse transcribing the individual mRNA samples and performing a template switching reaction to produce cDNA incorporating a barcode sequence; d) pooling and purifying the barcoded cDNA produced from the separate cells; e) amplifying the barcoded cDNA to generate a cDNA library comprising double-stranded cDNA; f) purifying the double-stranded cDNA; g) fragmenting the purified cDNA; h) purifying the cDNA fragments; and i) sequencing the cDNA fragments. In some alternative embodiments, the invention provides a method for gene profiling, comprising a) providing an isolated population of cells; b) releasing mRNA from the population of cells to provide one or more mRNA samples; c) reverse transcribing the one or more mRNA samples and performing a template switching reaction to produce cDNA incorporating a barcode sequence; d) pooling and purifying the barcoded cDNA; e) amplifying the barcoded cDNA to generate a cDNA library comprising double-stranded cDNA; f) purifying the double-stranded cDNA; g) fragmenting the purified cDNA; h) purifying the cDNA fragments; and i) sequencing the cDNA fragments.

In certain embodiments, the method further comprises separating a population of cells (e.g., by flow cytometry) to provide the plurality of single cells, for example, by separating them into a capture plate. In alternative embodiments, a population of cells can be sorted into a capture plate such that each well of the capture plate contains a smaller population of cells. Alternatively, cell lysate or RNA samples can be divided into a capture plate. In certain embodiments, the mRNA is released by cell lysis, for example, by freeze-thawing and/or contacting the cells with proteinase K. In certain embodiments, c) comprises contacting each individual mRNA sample with one or more nucleic acids as described above, for example i) a nucleic acid comprising a 5′ poly-isonucleotide sequence, an internal adapter sequence, and a 3′ guanosine tract, ii), a nucleic acid comprising a 5′ blocking group (e.g., biotin or an inverted nucleotide), an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine, or iii) both. In certain embodiments, c) is carried out with a reverse transcriptase enzyme, for example, a Moloney Murine Leukemia Virus (MMLV) reverse transcriptase such as SMARTscribe™ reverse transcriptase or SuperScript II™ reverse transcriptase or Maxima H Minus™ reverse transcriptase. In certain embodiments, the cDNA purification of d) is carried out with a Zymo-Spin™ column.

In certain embodiments, the method further comprises treating the barcoded cDNA with an exonuclease, such as with Exonuclease I. In certain embodiments, the amplification of e) utilizes an amplification primer comprising a 5′ blocking group, such as biotin or an inverted nucleotide. Exemplary amplification primers are 12 to 32 nucleotides in length, for example, 22 nucleotides in length (e.g., as in the amplification primer having the sequence of 5′-ACACTCTTTCCCTACACGACGC-3′ (SEQ ID NO: 2)). In certain embodiments, the purification off) may be carried out with magnetic beads, e.g., Agencourt AMPure XP magnetic beads (Beckman Coulter, #A63880), and/or may further comprise quantifying the purified cDNA. In certain embodiments, the single cells are provided in a capture plate of individual wells (e.g., a 384 well plate), each well comprising a single cell. In alternative embodiments, a population of cells is provided in a capture plate, each well comprising a population of cells. Alternatively, cell lysate or RNA samples can be provided in a capture plate. In should be understand throughout that when referring to identification of a particular sample, such as a sample in a well of a plate, that sample may be a single cell or some other sample, such as a lysate or bulk RNA. Thus, reference to a “well” or “sample” should be understood to refer to any of those types of samples. In certain embodiments, reference to “cell/well” or “well/cell” is similarly used to reflect that a sample may be a single cell or some other sample. When a sample is a single cell, identification of a well is equivalent to identification of a single cell. When the sample is something other than a single cell, identification of a well identifies the well in which that sample is provided but does not necessarily identify a single cell.

In certain embodiments, the fragmentation of g) utilizes a transposase, and may further utilize a first fragmentation nucleic acid and a second fragmentation nucleic acid, wherein the first fragmentation nucleic acid comprises a barcode sequence. An exemplary first fragmentation nucleic acid is 5′-CAAGCAGAAGACGGCATACGAGAT[i7]GTCTCGTGGGCTCGG-3′ (SEQ ID NO: 4), wherein [i7] represents a barcode sequence. In some embodiments, the [i7] sequence is four to 16 nucleotides in length, for example, eight nucleotides in length. In some embodiments, the [i7] sequence uniquely identifies a single population of nucleic acid species, for example, a population of nucleic acid species derived from a population of single cells from a capture plate. In some embodiments, the [i7] sequence is selected from: TCGCCTTA (SEQ ID NO: 5), CTAGTACG (SEQ ID NO: 6), TTCTGCCT (SEQ ID NO: 7), GCTCAGGA (SEQ ID NO: 8), AGGAGTCC (SEQ ID NO: 9), CATGCCTA (SEQ ID NO: 10), GTAGAGAG (SEQ ID NO: 11), CCTCTCTG (SEQ ID NO: 12), AGCGTAGC (SEQ ID NO: 13), CAGCCTCG (SEQ ID NO: 14), TGCCTCTT (SEQ ID NO: 15), and TCCTCTAC (SEQ ID NO: 16). In certain embodiments, the barcode sequence of the first fragmentation nucleic acid is different than the barcode sequence of the nucleic acid described in ii) above. In certain embodiments, the barcode sequence of the first fragmentation nucleic acid uniquely identifies a predetermined subset of cells, for example, a subset of cells contained in individual wells of a single capture plate. In further embodiments, the barcode sequence that uniquely identifies the predetermined subset of cells uniquely identifies the capture plate. In certain embodiments, the barcode sequence of the nucleic acid as described in ii) above uniquely identifies the cell within the predetermined subset of cells, which cell comprised the mRNA from which the barcoded cDNA of c) was produced. In further embodiments, the barcode sequence that uniquely identifies the cell within the predetermined subset of cells uniquely identifies an individual well in a capture plate, and in still further embodiments, the combination of the barcode sequence that uniquely identifies the predetermined subset of cells and the barcode sequence that uniquely identifies the cell within a predetermined subset of cells uniquely identifies the capture plate and the individual well which comprised the cell, which cell comprised the mRNA from which the barcoded cDNA of c) was produced. In certain embodiments, the barcode sequence of the first fragmentation nucleic acid is 4 to 20 nucleotides in length, for example, 6 nucleotides in length. In certain embodiments, the second fragmentation nucleic acid is a phosphorothioate bond-containing nucleic acid comprising an X1*X2*X3*X4*X5*3′ sequence, wherein * is a phosphorothioate bond. An exemplary second fragmentation nucleic acid is 48 to 68 nucleotides in length, e.g., 58 nucleotides in length, such as a second fragmentation nucleic acid with a sequence of 5′-AATGATACGGCGACCACCGAGATCTACACTCTTTCCCTACACGACGCTC TTCCG*A*T*C*T*-3′ (SEQ ID NO: 3).

In certain embodiments, the purification of h) is carried out with magnetic beads, and may optionally further comprise separating the magnetic-bead purified cDNA on an agarose gel, excising cDNA corresponding to 300 to 800 nucleotides in length, and purifying the excised cDNA. In certain embodiments, h) further comprises quantifying the purified cDNA. In certain embodiments, the sequencing of i) is carried out using RNA-seq. In certain embodiments, the method further comprises assembling a database of the sequences of the sequenced cDNA fragments of j), and may additionally comprise identifying the UMI sequences of the sequences of the database. In further embodiments, j) further comprises discounting duplicate sequences that share a UMI sequence, thereby assembling a set of sequences in which each sequence is associated with a unique UMI.

In certain embodiments, a) through h) are repeated before i) to produce a plurality of populations of cDNA fragments, and in particular embodiments, the populations of cDNA fragments are combined prior to i). In certain embodiments, the barcode sequence of the first fragmentation nucleic acid and the barcode sequence of the nucleic acid as described in ii) above are used to correlate the sequencing data with the predetermined subset of cells and the individual cell.

### BRIEF DESCRIPTION OF THE DRAWINGS

FIG. 1 depicts incomplete differentiation of human adipose tissue-derived stromal/stem cells (hASCs) in vitro. FIG. 1A: cells at day 0. FIG. 1B: cells at day 7 (i.e., on the seventh day after the cells were induced to differentiate). FIG. 1C: cells at day 14 (i.e., on the fourteenth day after the cells were induced to differentiate).

FIG. 2 depicts a flow chart of an exemplary method for single cell RNA sequencing.

FIG. 3 depicts how a single cell digital gene expression library was constructed, including barcode sequences incorporating sequencing primer sequences, indicated by arrows, and regions that anneal to their complementary oligonucleotides on a flow cell during sequencing (P5 and P7). N₆: cell/well barcode index; N₁₀: Unique Molecular Identifier (UMI). The sequencing primer with an i7 plate index is indicated by an arrow, and the two sequencing primers (read 1 and read 2) also are indicated by arrows.

FIG. 4 depicts a reduction in PCR bias through the use of Unique Molecular Identifier (UMI) sequences.

FIG. 5 depicts distributions of expression levels of the key marker genes FABP4 (FIG. 5A), SCD (FIG. 5B), LPL (FIG. 5C), and POSTN (FIG. 5D) during adipocyte differentiation. Particularly, FIG. 5 depicts the expression levels of gene across the cells/wells over time such that the position on the y axis shows the level of expression and the thickness of the bar shows the number of cells expressing at that level.

FIG. 6 depicts gene detection in single cells. Approximately 3,000 to 4,000 unique genes were detected per cell and approximately 15,000 unique genes were detected across all cells. Gene expression was reliably detected at approximately 25 to 50 transcripts per cell, although bursty transcription (transcription occurring in pulses rather than at a constant rate) introduced additional variation.

FIG. 7 depicts GAPDH detection at day 0. FIG. 7A depicts a histogram showing the distribution of GAPDH expression among cells profiled at day 0 as an exemplification of a transcriptional burst. FIG. 7B depicts genes associated with GAPDH. FIG. 7C provides a pictorial representation of the cell cycle. GAPDH is considered to be a housekeeping gene and often is used as a reference gene for normalization.

FIG. 8 depicts principal component analysis of an hASC population at day 0.

FIG. 9 depicts principal component analysis of an hASC population at day 0 (black) and day 1 (gray).

FIG. 10 depicts principal component analysis of an hASC population at day 0 (black) and day 2 (gray).

FIG. 11 depicts principal component analysis of an hASC population at day 0 (black) and day 3 (gray).

FIG. 12 depicts principal component analysis of an hASC population at day 0 (black) and day 7 (gray).

FIG. 13 depicts principal component analysis of an hASC population at day 0 (black) and day 14 (gray).

FIG. 14 depicts differentially expressed genes between day 0 (black) and day 14 (gray) hASC populations and between day 14 sub-populations.

FIG. 15 depicts the expression of adipocyte genes correlating with G1-arrest. Genes that had similar expression levels at Day 14 and Day 0 (FIG. 15A, label A) correspond to categories of genes involved in G-1 arrest (FIG. 15B, label A), indicating that those cells that did not fully differentiate may be stuck in the G0 phase. This reveals a correlation between differentiation state and cell cycle progression when gene expression is analyzed at the single cell level.

FIG. 16 depicts the process of adipocyte differentiation in mouse (3T3-L1) and human (hASC) stem cells, and that an absence of clonal expansion of hASCs may limit adipogenesis.

FIG. 17 depicts cell culture heterogeneity using single-cell sequencing. FIG. 17A depicts gene expression estimates from bulk cells compared to their corresponding means across single cell profiles. UPM: unique molecular identifier (UMI) counts for one gene per million UMI counts for all genes. FIG. 17B depicts the distribution of observed pairwise correlations (Pearson's r) between all pairs of genes that were detected in at least 10% of day 7 cells (n=4,038 genes), as compared to an estimated null distribution obtained by permuting the expression values of each gene across the same cells. FIGS. 17C and 17D depict single cell qRT-PCR validation and single molecule FISH validation, respectively, of the observed positive correlation between the LPL and G0S2 markers from separate cells also collected at day 7.

FIG. 18 depicts a comparison of RefSeq gene expression levels as estimated from the total number of raw aligned sequencing reads or the total number of unique UMIs. Each dot compares the mean raw counts across all profiled cells in the first time course (D1) to the mean UMI counts for the same gene. The raw and UMI counts are strongly correlated, but the UMI counts correct for a systematic bias in the raw expression levels of a subset of genes, which is likely caused by preferential PCR amplification or sequencing.

FIG. 19 depicts the relationship between the proportion of cells where a gene was detected (UMI count≧1) and its estimated expression level from bulk RNA profiling. Data is shown for day 0 of the D3 differentiation time course. Solid line: medians; top and bottom dotted lines: 90th and 10th percentiles, respectively. UPM=UMI counts for a gene per million UMI counts from all genes.

FIG. 20 depicts a comparison of single-molecule RNA sequencing (FIG. 20A) and single molecule FISH (smFISH, FIG. 20B) data for LPL and G0S2 during the D3 time course. Single-molecule RNA sequencing values are in UPM, while smFISH measurements are in mRNAs detected per cell. The smFISH data confirm the positive correlation between LPL and G0S2 after 7 days of differentiation. R: Pearson's correlation coefficient.

FIG. 21 depicts gene expression dynamics at single cell resolution. Each scatter plot depicts the first three principal components (PCs) of the initial hASC time course at the indicated time point (FIG. 21A: day 0; FIG. 21B: day 1; FIG. 21C: day 2; FIG. 21D: day 3; FIG. 21E: day 5; FIG. 21F: day 7; FIG. 21G: day 9; FIG. 21H: day 14). Black dots show cells collected at the indicated time point, while gray dots show cells collected at all previous time points. FIG. 21I depicts separately sorted cells with high and low lipid content from day 14 projected into the same PC space.

FIG. 22 depicts distributions of weights for the top four PCs in an initial hASC time course and a lipid-based sorting. To the right of the gene expression data, selected genes and gene sets associated with positive and negative weights are provided. Percentages indicate the ratio of the total variance in the data set captured by each PC. Horizontal lines within each set of boxes indicate medians, boxes indicate the 1st and 3rd quartiles, and whiskers indicate the ranges.

### DETAILED DESCRIPTION OF THE INVENTION

The present invention provides nucleic acids, kits, and methods for transcriptome-wide profiling at single cell resolution. In some embodiments, the invention provides Unique Molecular Identifiers (UMIs) (e.g., polynucleotides comprising UMIs) that specifically tag individual cDNA species as they are created from mRNA, thereby acting as a robust guard against amplification biases. Each UMI enables a sequenced cDNA to be traced back to a single particular mRNA molecule that was present in a cell. In some embodiments, the invention provides two levels of barcode-based multiplexing, allowing a sequenced cDNA to be traced to a particular cell from among a subset of cells. In some embodiments, the invention provides efficient transposon-based fragmentation, resulting in high yield cDNA libraries. In some embodiments, the invention provides sequencing of the 3′-end of mRNAs, limiting the sequencing coverage required to assess gene expression level of each single cell transcriptome. The methods allow the preparation of RNA-seq libraries in a manner that is not labor-intensive or time-consuming. Indeed, RNA-seq libraries of a thousand single cells can be easily prepared in two days. Any of the foregoing (or any of the nucleic acids, reagents, kits, and methods described herein may be provided and/or used alone or in any combination).

The foregoing is also applicable to populations of cells, cell lysates, tissue lysates, and/or extracted/purified RNA. For example, the invention also provides nucleic acids, kits, and methods for sequencing of extracted/purified RNA (bulk RNA sequencing) or for analysis of an isolated population of cells (e.g., from an isolated population of cells or a tissue; analysis of a cell or tissue lysate). In certain embodiments, any of the compositions, reagents, and methods described herein as applicable to single cells also are applicable to other sources of starting materials, such as extracted RNA, purified RNA, cell lysates, or tissue lysates, and such application is contemplated. In certain embodiments, any of the compositions, reagents, and methods described herein as applicable to extracted RNA, purified RNA, cell lysates or tissue lysates, also are applicable to single cells, and such application is contemplated.

The present invention provides improved nucleic acids, kits, and methods capable of transcriptome-wide profiling at single cell resolution of tens of thousands of cells simultaneously and cost-effectively (approximately $2 per sample, as compared to approximately $80 per sample with a current method). In certain embodiments, the methods and kits may include both customized nucleic acids and/or method steps that are themselves the subject of this application, as well as one or more commercially available reagents, kits, apparatuses, or method steps. The methods of the invention provide a number of distinct advantages over existing methods. Some current methods require a polyA addition step prior to sequencing, but this step can be eliminated through the use of a Moloney Murine Leukemia Virus reverse transcriptase. Moreover, full-length cDNA amplification can be carried out using the suppression PCR principle, thereby enriching full length cDNAs, and the method can be applied directly to cells rather than requiring RNA extraction first.

The methods of the invention also provide an advantage in that they utilize at least two barcode sequences rather than one, allowing for the simultaneous sequencing of at least 4,608 single-cell transcriptomes in a single lane, as compared to only 96 transcriptomes in current methods. Still further, optimization of reaction volumes can conserve expensive reagents, such as the reverse transcriptase enzyme, reducing costs. Additionally, by utilizing 3′ end digital sequencing, less sequencing coverage is needed to determine gene expression levels, further reducing costs.

The methods of the invention provide an advantage over current methods targeting the 3′ end of mRNA that use linear mRNA amplification. Linear mRNA amplification is time-consuming compared to template switching/suppression PCR amplification. Linear mRNA amplification also is labor-intensive and limits the number of cells that can be processed to approximately 50 cells per day by a single person. By contrast, the methods of the invention can accommodate 384 cells in a single plate, allowing a single person to easily process up to 1152 cells per day.

The use of UMIs also provides a distinct advantage over typical single-cell RNA-seq methods. Because of the very low starting amount of RNA in a single cell, several amplification steps are required during the process of the RNA-seq library preparation, and the UMIs protect against amplification biases.

The methods of the invention utilizing a transposase-based sequencing library preparation have the added advantage of eliminating a number of labor-intensive and costly steps in library preparation, including magnetic bead immobilization, separate fragmentation, end repair, dA-tailing, and adaptor ligation. By eliminating the separate steps of chemical fragmentation and its purification, end repair, dA-tailing and adapter ligation, labor and cost are reduced, and the yield is much higher than with other techniques because there are fewer purification steps (during which material can be lost) and because this method to tag the fragment is much more efficient than by ligation with a regular ligase. Because less material is lost in the process, the methods of the invention can start with a much lower amount of starting cDNA. This is beneficial because even when combining and amplifying cDNA from 384 cells, there is often a low starting amount of cDNA to begin the library preparation.

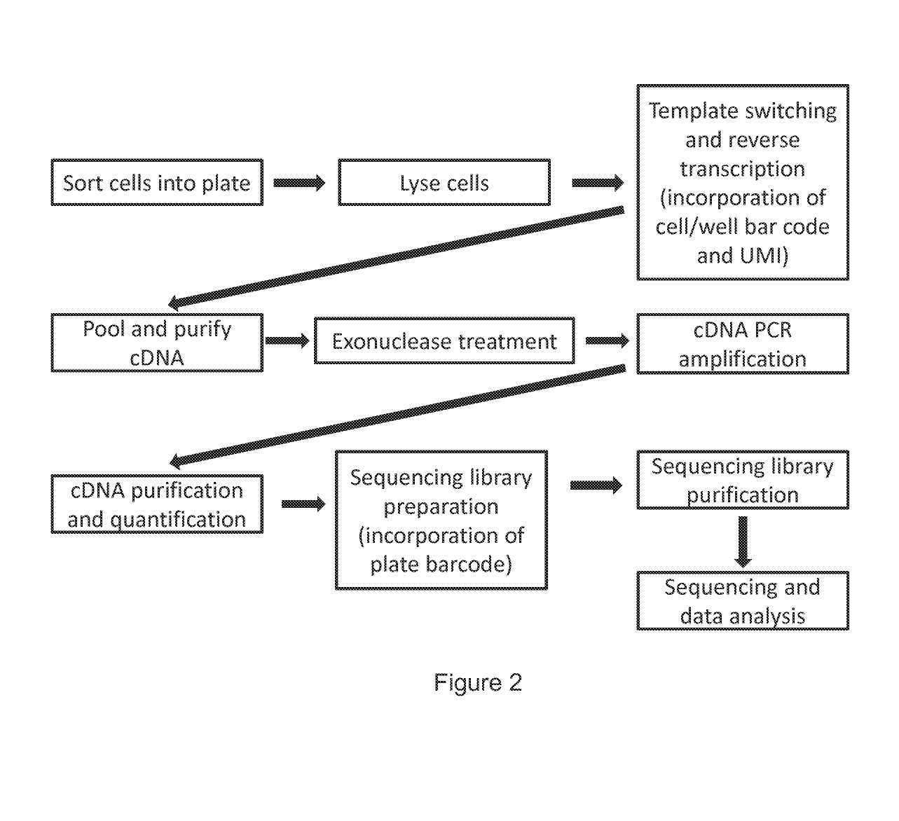

The invention provides methods that are advantageous based on a number of improvements to existing methods. A typical method provided by the invention is depicted in FIG. 2, and starts with preparing a capture plate for cell sorting. Cells are then sorted into the plate (e.g., by fluorescence activated cell sorting), after which the plate may be frozen down for storage. For single cell analysis, one cell is sorted into each well of the plate. One advantage of the nucleic acids provided herein is that the use of various barcodes permits the end user to correlate transcript expression back to a particular well and plate, and thus to a specific cell evaluated. To lyse the cells, the plate can, in certain embodiments, be thawed from its frozen state. Optionally, a proteinase or protease, such as proteinase K, is added to the cells to increase the efficiency of the lysis. If performing bulk RNA-seq, the cell sorting and individual cell lysis steps can be skipped, as the starting material is already RNA. If the starting material is a population of cells, the population can be divided into a multi-well plate in preparation for lysis. Or, if the starting material is a lysate prepared from a population of cells or tissues, cell or tissue lysis may optionally occur in a prior step before introduction into the well and then lysate itself may be added to each well of a multi-well plate. For example, a population of cells can be sorted into lysis buffer and lysed (e.g., by freeze-thawing, proteinase K treatment, or a combination thereof) before the lysate is added to the plate. The next steps are to reverse transcribe the mRNA that has been released from the cells and to perform a template switching step. The reverse transcription and template switching can be performed using the nucleic acids of the invention, which efficiently perform these steps. For example, a cDNA synthesis primer comprising a 5′ blocking group, an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine, can be used for reverse transcription. Here, the 5′ blocking group is used to ensure the correct directionality of cDNA synthesis and the adapter sequence provides a sequence annealing to a sequencing primer, so the first sequencing read will contain the barcode and UMI sequences. Part of the adapter sequence also is used during the suppression PCR. The barcode sequence is used to track which well (and, thus, which cell) a particular cDNA was generated from. In bulk RNA-seq and lysate sequencing embodiments, a barcode can provide a reference for (and, thus, a way to identify) the sample or the pool (e.g., the well) rather than a single cell. Alternatively, a UMI can be used in bulk RNA-seq and lysate sequencing to identify the transcript and the i7 primer (which, in other embodiments, typically contains the barcode for the plate, e.g., for plate indexing—sometimes referred to as the plate barcode or the index) identifies the sample or pool (e.g., the well) rather than the single cell. In these embodiments, the UMI can be, for example, a 16mer UMI. Thus, in certain embodiments, a combination of one or more barcodes and a UMI is used. In other embodiments, a UMI is used either alone or with a single barcode. In either way, the methods and compositions provide a mechanism for identifying where a particular transcript came from. In certain embodiments, i7 is used for plate indexing (e.g., it is a barcode to identify a particular plate). In other embodiments, i7 serves as a sample barcode. The UMI provides a way to trace each cDNA produced to a particular mRNA derived from a cell/sample. The complementarity sequence anneals to the mRNA, for example, to the poly(A) tail of an mRNA, although it also could anneal to a specific target sequence, such as the sequence of a particular mRNA, instead. The 3′ dinucleotide sequence target the extremity of the polyA tail, the last two bases of the mRNA before the polyA tail. These two final nucleotides prevent the nucleic acid from annealing elsewhere within the polyA tail, which can be as 10 ng as 250 bp in length. If the nucleic acid were to bind elsewhere, one would not be able to directly access the useful sequence information of the transcript. A template-switching oligonucleotide comprising a 5′ poly-isonucleotidecytosine-isoguanosine-isocytosine sequence, an internal adapter sequence, and a 3′ guanosine tract can be used in the template switching step. The 5′ poly-isonucleotidecytosine-isoguanosine-isocytosine sequence provides non-standard base pairs in the template switching oligo to prevent background cDNA synthesis. These nucleotide isomers inhibit reverse transcriptase, such as MMLV reverse transcriptase, from extending the cDNA beyond the template switching adapter, thus increasing cDNA yield by reducing formation of concatemers of the template switching adapter. The adapter sequence provides the sub sequence required for the suppression PCR, and the 3′ guanosine tract is used to anneal to a polycytosine tract generated at the 3′ end of the first strand of cDNA synthesized. These steps are useful in incorporating a barcode and a UMI into the resulting cDNAs. The barcode introduced here helps track the individual well (and, therefore, cell/sample) that a cDNA population came from, while the UMI is unique for each mRNA that produces a cDNA. Thus, the population of UMIs incorporated into the cDNAs provide a molecular “snapshot” of the mRNA population of the cell or sample at the time of lysis, because subsequent amplification steps do not alter the number of UMIs, making it possible to trace back each cDNA sequenced later to a particular mRNA released from the cell/sample. The template switching step is selective for the creation of full-length cDNAs.

After reverse transcription and template switching, the wells can be pooled together and purified, followed by treatment with an exonuclease such as Exonuclease I. Without the exonuclease treatment, such as Exonuclease I treatment, the primer used for the suppression PCR can bind to the remaining adapters that are in excess from the template switching reaction, so the addition of an exonuclease, such as Exonuclease I, improves results. The cDNAs then are amplified (e.g, via PCR), followed by subsequent purification and quantification steps. Next, the library is prepared for sequencing by fragmentation, e.g., with a transposase-based fragmentation system. This step also introduces a second bar code to the cDNAs, this second bar code being specific for the capture plate from which the cDNAs were pooled. Thus, each cDNA will have a bar code for both the plate and the well from which it was derived, allowing simultaneous processing of a large number of samples, in which each individual sequence can be traced back to a single mRNA of a specific cell (or, in the case of another type of sample, to be traced back to a well containing a cell or tissue lysate sample, a purified RNA sample, or the like). The library then can be purified, selected for appropriate size fragments, assessed for quantity and quality, and sequenced (e.g., by RNA-seq such as the Illumina HiSeg™ (Catalog # SY-401-2501) or MiSeg™ (Catalog # SY-410-1003) systems). The sequencer can handle various read lengths and either single-end or paired-end sequencing. The libraries can be run in a way that matches with the read length required to read each barcode and obtains enough information from the sequence of the cDNA to identify from which gene it was coming from. For example, 17 cycles can be run for read 1 (see above) to read first the 6 bp well/cell barcode and the 10 bp of UMI. This is then followed by 9 cycles to read the 8 bp i7 plate index. Finally, 46 cycles are, in certain embodiments, run on the other strand to read the cDNA/gene sequence. The machine allows the operator to set up a custom run for which they decide the read length for each portion for which sequence is to be obtained. This sequencing design allows an individual to decipher all the information while using the smaller/cheapest kit to meet their needs (e.g., 50 cycle kit that actually contains enough reagents for 74 cycles). Alternatively, an individual could run more cycles to get longer stretches of cDNA.

Before sequencing, samples from multiple capture plates can be combined without losing the identity of each cDNA in the mixture because of the two barcode sequences. Thus, the data can be deconvoluted after sequencing to determine the UMI of each particular cDNA and the well and plate it came from via the barcodes. This is advantageous because it allows a researcher to run many more samples together than would otherwise be possible, and to do so with less cost and labor.

### DEFINITIONS

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The term “diagnosis” as used herein refers to methods by which the skilled artisan can estimate and/or determine whether or not a patient is afflicted with a given disease or condition. The skilled worker often makes a diagnosis based on one or more diagnostic indicators. Exemplary diagnostic indicators may include the manifestation of symptoms or the presence, absence, or change in one or more markers for the disease or condition. A diagnosis may indicate the presence or absence, or severity, of the disease or condition.

The term “prognosis” is used herein to refer to the likelihood of the progression or regression of a disease or condition, including likelihood of the recurrence of a disease or condition.

As used herein, “treating” a disease or condition refers to taking steps to obtain beneficial or desired results, including clinical results. Beneficial or desired clinical results include, but are not limited to, reduction, alleviation or amelioration of one or more symptoms associated with the disease or condition.

As used herein, “administering” or “administration of” a compound or an agent to a subject can be carried out using one of a variety of methods known to those skilled in the art. For example, a compound or an agent can be administered orally, intravenously, arterially, intradermally, intramuscularly, intraperitoneally, subcutaneously, ocularly, sublingually, intranasally, intraspinally, intracerebrally, and transdermally. A compound or agent can appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods. Administration of a compound may include both direct administration, including self-administration, and indirect administration, including the act of prescribing a drug. For example, a physician who instructs a patient to self-administer a therapeutic agent, or to have the agent administered by another, and/or who provides a patient with a prescription for a drug has administered the drug to the patient.

The term “nucleic acid” refers to DNA molecules (e.g., cDNA or genomic DNA), RNA molecules (e.g., mRNA), DNA-RNA hybrids, and analogs of the DNA or RNA generated using nucleotide analogs. The nucleic acid molecule can be a nucleotide, oligonucleotide, double-stranded DNA, single-stranded DNA, multi-stranded DNA, complementary DNA, genomic DNA, non-coding DNA, messenger RNA (mRNA), microRNA (miRNA), small nucleolar RNA (snoRNA), ribosomal RNA (rRNA), transfer RNA (tRNA), small interfering RNA (siRNA), heterogeneous nuclear RNAs (hnRNA), or small hairpin RNA (shRNA).

As used herein, a “profile” of a transcriptome or portion of a transcriptome can refer to any sequencing or gene expression information concerning the transcriptome or portion thereof. This information can be either qualitative (e.g., presence or absence) or quantitative (e.g., levels or mRNA copy numbers). In some embodiments, a profile can indicate a lack of expression of one or more genes.

The term “cDNA library” refers to a collection of complementary DNA (cDNA) fragments. A cDNA library may be generated from the transcriptome of a single cell or from a plurality of single cells. cDNA is produced from mRNA found in a cell and therefore reflects those genes that have been transcribed for subsequent protein expression.

As used herein, a “plurality” of cells refers to a population of cells and can include any number of cells to be used in the methods described herein. For example, a plurality of cells includes at least 10 cells, at least 25 cells, at least 50 cells, at least 100 cells, at least 200 cells, at least 500 cells, at least 1,000 cells, at least 5,000 cells, or at least 10,000 cells. In some embodiments, a plurality of cells includes from 10 to 100 cells, from 50 to 200 cells, from 100 to 500 cells, from 100 to 1,000 cells, or from 1,000 to 5,000 cells.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Cells can be cultured cells or cells from a dissociated tissue, and can be fresh or preserved in a preservative buffer such as RNAprotect. Furthermore, in general, cells from any population can be used in the methods, such as a population of prokaryotic or eukaryotic single-celled organisms including bacteria or yeast. In some aspects of the invention, the method of preparing the cDNA library can include the step of obtaining single cells. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, such that each single cell is placed in a single well.

As used herein, an “oligonucleotide” or “polynucleotide” refers to a polymeric form of nucleotides of any length, either deoxyribonucleotides or ribonucleotides or analogs thereof. Polynucleotides can have any three-dimensional structure and can perform any function. Exemplary polynucleotides include a gene or gene fragment (e.g., a probe or primer), exons, introns, messenger RNA (mRNA), transfer RNA, ribosomal RNA, ribozymes, cDNA, recombinant polynucleotides, branched polynucleotides, plasmids, vectors, isolated DNA or RNA of any sequence, and nucleic acid probes and primers. A polynucleotide can comprise modified nucleotides, such as isonucleotides, methylated nucleotides, and other nucleotide analogs. The term also refers to both double- and single-stranded molecules. A polynucleotide is composed of a specific sequence of four nucleotide bases: adenine (A), cytosine (C), guanine (G), and thymine (T). Uracil (U) substitutes for thymine when the polynucleotide is RNA. The sequence can be input into databases in a computer having a central processing unit and used for bioinformatics applications such as functional genomics and homology searching.

As used herein, a “primer” is a polynucleotide that hybridizes to a target or template that may be present in a sample of interest. After hybridization, the primer promotes the polymerization of a polynucleotide complementary to the target, for example in a reverse transcription or amplification reaction.

### Cell Sorting and Lysis

Methods for selecting or sorting cells are well established, and in some embodiments include, but are not limited to, fluorescence-activated cell sorting (FACS), micromanipulation, manual sorting, and the use of semi-automated cell pickers. Individual cells can be individually selected based on features detectable by observation (e.g., by microscopic observation). Exemplary features can include location, morphology, and reporter gene expression. A population of cells can be sorted to provide a subpopulation or a predetermined subset of cells. In some embodiments, the population, subpopulation, or predetermined subset can be sorted to provide single cells. In some embodiments, the cells are sorted into a capture plate. Capture plates can comprise a number of wells into which the cells are sorted, for example, 24 wells, 96 wells, 384 wells, or 1536 wells. In some embodiments, a population of cells is lysed without sorting. The population of cells can be, for example, a tissue sample. In certain embodiments, the population of cells is an isolated population of cells. In such embodiments, the starting material for further analysis may be, for example, a cell or tissue lysate or bulk purified or extracted RNA. In such embodiments, cells can be divided into the wells of a plate without sorting. In particular embodiments, the amount of material in each well is normalized with respect to the other wells so as to provide similar sequencing coverage across a plate.

To release mRNA from cells, the cells may be lysed. Cells may be lysed by any number of known techniques. Exemplary cell lysis techniques include freeze-thawing, heating the cells, using a detergent or other chemical method, or a combination thereof. Techniques minimizing degradation of the released mRNA are preferred. Likewise, techniques preventing the release of nuclear chromatin are preferred. For example, heating the cells in the presence of Tween-20 is sufficient to lyse cells while minimizing genomic contamination from nuclear chromatin. In certain embodiments, cells are lysed using freeze-thawing. In some embodiments, a proteinase or protease, such as proteinase K, is added to the lysis reaction to increase the efficiency of lysis. In certain embodiments, cells are lysed using freeze-thawing optionally supplemented with addition of proteinase K.

As noted above, cell lysis may be of single cells already sorted into individual wells of a plate. Alternatively, lysis of populations of cells may be performed and the starting material for further sequence analysis may be a cell or tissue lysate made from a plurality of cells and then aliquoted to wells of a plate. Regardless of starting material, in certain embodiments, following lysis the material may be stored at a suitable temperature, such as −80° C., prior to further use.

### Reverse Transcription and Template Switching

In some embodiments, cDNA is synthesized from mRNA through the process of reverse transcription. Reverse transcription can be performed directly on cell lysates (for example, a cell lysate prepared as described above), by adding a reaction mix for reverse transcription directly to the cell lysate. In alternative embodiments, the total RNA or mRNA can be purified after cell lysis, for example through the use of column based (e.g., Qiagen RNeasy Mini kit Cat. No. 74104, ZymoResearch Direct-zol RNA Cat. No. R2050) or magnetic bead purification (e.g., Agencourt RNAClean XP, Cat. No. A63987). Methods for reverse transcription of mRNA to cDNA are well established in the art. In some embodiments, the reverse transcription is combined with a template switching step to improve the yield of longer (e.g., full length) cDNA molecules. In certain embodiments, the reverse transcriptase used has tailing or terminal transferase activity, and synthesizes and anchors first-strand cDNA in one step. In certain embodiments, the reverse transcriptase is a Moloney Murine Leukemia Virus (MMLV) reverse transcriptase, for example, SMARTscribe™ (Clontech, Cat. No. 639536) reverse transcriptase, SuperScript II™ reverse transcriptase (Life Technologies, Cat. No. 18064-014), or Maxima H Minus™ reverse transcriptase. (Thermo Scientific, Cat. No. EP0753).

Template switching introduces an arbitrary sequence at the 3′ end of the cDNA that is designed to be the reverse complement to the 3′ end of a cDNA synthesis primer. In some embodiments, the synthesis of the first strand of the cDNA can be directed by a cDNA synthesis primer (CDS) that includes an RNA complementary sequence (RCS). In some embodiments, the RCS is at least partially complementary to one or more mRNA species in an individual mRNA sample, allowing the primer to hybridize to at least some mRNA species in a sample to direct cDNA synthesis using the mRNA as a template. The RCS can comprise oligo (dT) sequence that binds to many mRNA species, or it can be specific for a particular mRNA species, for example, by binding to an mRNA sequence of a gene of interest. Alternatively, the RCS can comprise a random sequence, such as random hexamers. To avoid the CDS self-priming, a non-self-complementary sequence can be used.

A template-switching oligonucleotide that includes a portion which is at least partially complementary to a portion of the 3′ end of the first strand of cDNA generated by the reverse transcription can also be used in the methods of the invention. Because the terminal transferase activity of reverse transcriptase typically causes the incorporation of two to five cytosines at the 3′ end of the first strand of cDNA synthesized, the first strand of cDNA can include a plurality of cytosines, or cytosine analogues that base pair with guanosine, at its 3′ end to which the template-switching oligonucleotide with a 3′ guanosine tract can anneal. During the template switching step, the template-switching oligonucleotide is extended to form a double stranded cDNA. Thus, in some embodiments, a template-switching oligonucleotide can include a 3′ portion comprising a plurality of guanosines or guanosine analogues that base pair with cytosine. Exemplary guanosines or guanosine analogues include, but are not limited to, deoxyriboguanosine, riboguanosine, locked nucleic acid-guanosine, and peptide nucleic acid-guanosine. The guanosines can be ribonucleosides or locked nucleic acid monomers. A locked nucleic acid is an RNA nucleotide wherein the ribose moiety has been modified with an extra bridge connecting the 2′ oxygen and the 4′ carbon. A peptide nucleic acid is an artificially synthesized polymer similar to DNA or RNA, wherein the backbone is composed of repeating N-(2-aminoethyl)-glycine units linked by peptide bonds.

In some embodiments, the reverse transcription and template switching comprise contacting an mRNA sample with two nucleic acid primers. In certain embodiments, the first nucleic acid primer (e.g., a template-switching oligonucleotide) comprising a 5′ poly-isonucleotidecytosine-isoguanosine-isocytosine sequence, an internal adapter sequence, and a 3′ guanosine tract. In certain embodiments, the 5′ poly-isonucleotide sequence comprises an isocytosine, or an isoguanosine, or both. In certain embodiments, the 5′ poly-isonucleotide sequence comprises an isocytosine-isoguanosine-isocytosine sequence. Incorporating non-natural nucleotides, such as an isocytosine or an isoguanosine into template-switching primers can reduce background and improve cDNA synthesis (Kapteyn et al., BMC Genomics. 11:413 (2010)). In some embodiments, the 3′ guanosine tract comprises two, three, four, five, six, seven, eight, nine, ten, or more guanosines. In certain embodiments, the 3′ guanosine tract comprises three guanosines. In some embodiments, the adapter sequence is 12 to 32 nucleotides in length, for example, 22 nucleotides in length. In particular embodiments, the internal adapter sequence is 5′-ACACTCTTTCCCTACACGACGC-3′ (SEQ ID NO: 1). In particular embodiments, the sequence of the first primer is 5′-iCiGiCACACTCTTTCCCTACACGACGCrGrGrG-3′ (SEQ ID NO: 17)(e.g., 1 μM,) wherein iC represents isocytosine (iso-dC), iG represents isoguanosine, and rG represents RNA guanosine.

In certain embodiments, the second nucleic acid primer (e.g., a cDNA synthesis primer) comprises a 5′ blocking group, an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine. Optionally, to sequence bulk RNA or lysates, the bar code can be omitted from the cDNA synthesis primer and an extra 6 base pairs can be added to the UMI sequence. In particular embodiments, the 5′ blocking group is selected from biotin, an inverted nucleotide (e.g., inverted dideoxy-T), a fluorophore, an amino group, and iso-dG or isodC. In particular embodiments, the internal adapter sequence is 23 to 43 nucleotides in length, for example, 33 nucleotides in length. In particular embodiments, the internal adapter sequence is 5′-ACACTCTTTCCCTACACGACGC-3′ (SEQ ID NO: 1). In particular embodiments, the barcode sequence is 4 to 20 nucleotides in length, for example, 4, 5, 6, 7, 8, 9, 10, 11, 12, 13, 14, 15, 16, 17, 18, 19, or 20 nucleotides in length. In particular embodiments, the UMI sequence is 6 to 20 nucleotides in length, for example, 6, 7, 8, 9, 10, 11, 12, 13, 14, 15, 16, 17, 18, 19, or 20 nucleotides in length. In particular embodiments, the complementarity sequence is a poly(T) sequence. In particular embodiments, the complementarity sequence is 20 to 40 nucleotides in length, for example, 20, 21, 22, 23, 24, 25, 26, 27, 28, 29, 30, 31, 32, 33, 34, 35, 36, 37, 38, 39, or 40 nucleotides in length. In specific embodiments, the second nucleic acid primer is 5′-/5Biosg/ACACTCTTTCCCTACACGACGCTCTTCCGATCT[BC6] NNNTTTTTTTTTTTTTTTTTTTTTTTTTTTTTTVN-3′ (SEQ ID NO: 18), wherein 5Biosg represents 5′ biotin; V represents a nucleotide selected from A, G, and C; the 3′ N represents a nucleotide selected from A, G, C, and T; [BC6] represents a 6 base pair barcode sequence; and the (N)10 after the barcode sequence represents a Unique Molecular Identifier (UMI) sequence. In these primers, the barcodes may be designed so that each barcode sequence differs from the barcodes of all other primers by at least two nucleotides, so that a single sequencing error cannot lead to the misidentification of the barcode.

The UMI sequences provide a robust guard against amplification biases. More particularly, each UMI is present only once in a population of second nucleic acid primers. Thus, each UMI is incorporated into a unique cDNA sequence generated from a cellular mRNA, and any subsequent amplification steps will not alter the one UMI to one mRNA ratio. In certain embodiments, the UMI sequence, rather than being 10 nucleotides in length, is 5, 6, 7, 8, 9, 11, 12, 13, 14, 15, 16, 17, 18, 19, 20, 21, 22, 23, 24, 25, 26, 27, 28, 29, 30, or more nucleotides in length. The length should be selected to provide sufficient unique sequences for the population of cells to be tested (preferably with at least two nucleotide differences between any pair of UMIs), preferably without adding unnecessary length that increases sequencing cost.

Barcode sequences enable each cDNA sample generated by the above method to have a distinct tag, or a distinct combination of tags, such that once the tagged cDNA samples have been pooled, the tag can be used to identify the single cell from which each cDNA sample originated. Thus, each cDNA sample can be linked to a single cell, even after the tagged cDNA samples have been pooled and amplified. In other words, the use of the foregoing nucleic acids permits deconvolution of pooled data to single cell/well resolution. This is particularly advantageous for facilitating the application of this technology to screening assays.

In some embodiments, a nucleic acid useful in the invention can contain a non-natural sugar moiety in the backbone, for example, sugar moieties with 2′ modifications such as addition of a halogen, alkyl-substituted alkyl, SH, SCH₃. OCN, Cl, Br, CN, CF₃, OCF₃, SO₂CH₃, OSO₂, NO₂, N₃, or NH₂. Similar modifications also can be made at other positions on the sugar. Nucleic acids, nucleoside analogs or nucleotide analogs having sugar modifications can be further modified to include a reversible blocking group, a peptide linked label, or both. In those embodiments comprising a 2′ modification, the base can have a peptide-linked label.

A nucleic acid useful in the invention also can include native or non-native bases. In some embodiments, a native deoxyribonucleic acid can have one or more bases selected from adenine, thymine, cytosine, and guanine, and a ribonucleic acid can have one or more bases selected from uracil, adenine, cytosine, and guanine Exemplary non-native bases include, but are not limited to, inosine, xanthine, hypoxanthine, isocytosine, isoguanosine, 5-methylcytosine, 5-hydroxymethyl cytosine, 2-aminoadenine, 6-methyl adenine, 6-methyl guanine 2-propyl guanine, 2-propyl adenine, 2-thiothymine, 2-thiocylosine, 5-propynyl uracil, 5-propynyl cytosine, 6-azo uracil, 6-azo cytosine, 6-azo thymine, 4-thiouracil, 8-halo adenine, 8-halo guanine, 8-amino adenine, 8-amino guanine, 8-thiol adenine, 8-thiol guanine, 8-thioalkyl adenine, 8-thioalkyl guanine, 8-hydroxyl adenine, 8-hydroxyl guanine, 5-halo substituted uracil, 5-halo substituted cytosine, 7-methylguanine, 7-methyladenine, 8-azaguanine, 8-azaadenine, 7-deazaguanine, 7-deazaadenine, 3-deazaguanine, and 3-deazaadenine. In certain embodiments, isocytosine and isoguanosine may reduce non-specific hybridization. In some embodiments, a non-native base can have universal base pairing activity, wherein it is capable of base-pairing with any other naturally occurring base, e.g., 3-nitropyrrole and 5-nitroindole.

cDNA Pooling and Purification

In some embodiments, after reverse transcription and template switching have been used to generate cDNA, the cDNA is pooled together. For example, a population of cells can be individually sorted into the wells of a tray, lysed, and undergo reverse transcription and template switching. These cDNAs then can be pooled and purified. In certain embodiments, the cDNA is purified through a column-based purification method, e.g., with a DNA Clean & Concentrator-5 column (Zymo Research, #D4013).

### Exonuclease Treatment

In some embodiments, pooled cDNAs are treated with an exonuclease (e.g., Exonuclease I) to degrade any primers remaining from the reverse transcription and template switching steps. This prevents possible interference by these primers in subsequent amplification.

### Amplification

As used herein, the term “amplification” or “amplifying” refers to a process by which multiple copies of a particular polynucleotide are formed, and includes methods such as the polymerase chain reaction (PCR), ligation amplification (also known as ligase chain reaction, or LCR), and other amplification methods. In some embodiments, amplification refers specifically to PCR. Amplification methods are widely known in the art. In general, PCR refers to a method of amplification comprising hybridization of primers to specific sequences within a DNA sample and amplification involving multiple rounds of annealing, elongation, and denaturation using a DNA polymerase. The resulting DNA products are then often screened for a band of the correct size. The primers used are oligonucleotides of appropriate length and sequence to provide initiation of polymerization. Reagents and hardware for conducting amplification reactions are widely known and commercially available. Primers useful to amplify sequences from a particular gene region are sufficiently complementary to hybridize to target sequences. Nucleic acids generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules. The stringency of hybridization is influenced by hybridization conditions, such as temperature and salt. In the context of amplification, these parameters can be suitably selected.

In some embodiments, cDNA created by reverse transcription and template switching, and optionally treated with an exonuclease, is amplified to provide more starting material for sequencing. cDNA can be amplified by a single primer with a region that is complementary to all cDNAs, e.g., an adapter sequence. In certain embodiments, the primer has a 5′ blocking group such as biotin. An exemplary primer is as follows: 5′-/5Biosg/ACACTCTTTCCCTACACGACGC-3′ (wherein 5Biosg represents 5′ biotin) (SEQ ID NO: 19). One exemplary amplification reaction uses cDNA; PCR buffer, such as 10× Advantage 2 PCR buffer; dNTPs; the DNA primer 5′-/5Biosg/ACACTCTTTCCCTACACGACGC-3′ (SEQ ID NO: 19); Polymerase Mix, such as Advantage 2 Polymerase Mix; and Water, such as nuclease-free water, and is (in certain embodiments) performed using the following program: 95° C. for 1 minute; 18 cycles of a) 95° C. for 15 seconds, 65° C. for 30 seconds, 68° C. for 6 minutes, and 72° C. for 10 minutes (followed by an optional hold period at 4° C.). In certain bulk RNA-seq and lysate sequencing embodiments, this amplification reaction may be modified to use fewer than 18 cycles, e.g., 10 cycles. One exemplary amplification reaction uses 204 of cDNA; 5 μL of 10× Advantage 2 PCR buffer; 1 μL of dNTPs; 1 μL of the DNA primer 5′-/5Biosg/ACACTCTTTCCCTACACGACGC-3′ (SEQ ID NO: 19) (10 μM, Integrated DNA Technologies); 1 μL of the Advantage 2 Polymerase Mix; and 22 μL of Nuclease-Free Water, and is optionally performed using the following program: 95° C. for 1 min; 18 cycles of a) 95° C. for 15 sec, 65° C. for 30 sec, 68° C. for 6 min, and 72° C. for 10 min (followed by an option hold period at 4° C.). However, the skilled worker will appreciate that amplification conditions may be adjusted depending on the exact primer and template being used.

### Nucleic Acid Purification and Quantification

Nucleic acid purification (e.g., cDNA purification) is well known in the art. In some embodiments, a nucleic acid (e.g., cDNA) is purified with a spin-based column, such as those commercially available from Zymo Research™ (DNA Clean & Concentrator™-5, Cat. No. D4013) or Qiagen™ (MinElute PCR purification kit. Cat. No. 28004). In particular embodiments, the spin column is a column lacking a physical ring, for example the ring found in Qiagen™ columns, allowing elution of the purified nucleic acid in a lower volume than would be possible in a spin column with a ring. In some embodiments, a nucleic acid (e.g., cDNA, such as in a cDNA library), is purified using magnetic beads. Magnetic bead purification systems are well known and include, for example, the Agencourt AMPure XP™ system (Beckman Coulter, Cat. No. A63881). In some embodiments, a nucleic acid (e.g., cDNA, such as in a cDNA library) is purified after being run on a gel. Gel extraction purification kits are well known, and include, for example, the MinElute Gel Extraction Kit™ (Qiagen, Cat. No. 28604).

### Sequencing Library Preparation

In some embodiments, a cDNA library for sequencing is fragmented prior to the sequencing. A cDNA library can be fragmented by any known method, for example, mechanical fragmentation or a transposase-based fragmentation such as that used in the Nextera™ system (e.g., the Illumina Nextera XT DNA Sample Preparation Kit Cat. No. FC-131-1096 or the Nextera DNA Sample Preparation Kit Cat. No. FC-121-1031). Fragmentation via a transposase-based system has the benefit of being able to incorporate into the fragments barcode sequences that facilitate identification of the fragments. In some embodiments, a barcode sequence introduced during preparation of a cDNA library for sequencing is specific for a predetermined set of cells. This predetermined set of cells can be a subset of a larger set of cells. For example, a tissue biopsy can be sorted into a set of cells to be further sorted into single cells in a capture plate for gene profiling. If a bulk lysate or population of cells is being used as a starting material rather than a single cells that have been sorted, a barcode sequence may, in certain embodiments, not be necessary in this step if a barcode already has been incorporated into the cDNA library in previous steps. However, a plate barcode still could be used to multiplex a high number of samples even for purified RNA/lysates.

### Sequencing Library Quality Assessment

In some embodiments, a cDNA library for sequencing is quantified and evaluated for quality prior to the sequencing to ensure that the library is of sufficient quantity and quality to yield positive results from sequencing. For example, a cDNA library can be quantified using a fluorometer and analyzed for quantity and average size through the use of a number of commercially available kits. The 2 main metrics for quality are the concentration of the library (which needs to be sufficient for loading on the sequencer) and the length of the cDNA fragments to be sequenced. Size selection is performed on a gel to enrich for fragments of the correct size. The gel itself gives an idea of the quality of the library. The final extracted library can be run on an Agilent Bioanalyzer (Cat. No. G2940CA) to obtain the size distribution for the cDNA fragments.

### Sequencing

As used herein, “sequencing” refers to any technique known in the art that allows the identification of consecutive nucleotides of at least part of a nucleic acid. Exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (MPSS), sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and a combination thereof. In some embodiments, sequencing comprises detecting a sequencing product using an instrument, for example but not limited to an ABI PRISM™ 377 DNA Sequencer, an ABI PRISM™ 310, 3100, 3100-Avant, 3730, or 3730xI Genetic Analyzer, an ABI PRISM™ 3700 DNA Analyzer, or an Applied Biosystems SOLiD™ System (all from Applied Biosystems), a Genome Sequencer 20 System (Roche Applied Science), or a mass spectrometer. In certain embodiments, sequencing is performed on Illumina Hiseq or MiSeq paired-end flow cells.

### Data Analysis

As described herein, one major advantage of the nucleic acids, methods, and kits of the invention is that samples can be pooled and sequenced rather than needing to be sequenced individually. Sequencing products can be traced not only to a single plate of cells from which it came, but also to a single cell (e.g., a well) and, indeed, a single cellular transcript. This deconvolution of sequencing data can be achieved through the use of barcode and UMI sequences. In some embodiments, sequencing is combined with 3′ digital gene expression to provide a number of counts for a particular sequence or sequences (e.g., cDNAs containing a particular combination of bar codes and a UMI). In some embodiments, each fragment of each transcript is sequenced and then counted for how many fragments of each transcript have been sequenced. In these embodiments, the computed gene expression should be normalized based on the length of a given transcript because a longer transcript will have a greater chance of having one of its fragments sequenced. However, full transcript sequencing typically requires more sequencing coverage than DGE, for which only the 3′ end needs to be sequenced.

### Kits

In some embodiments, the invention provides a kit comprising a plurality of the one or both of the reverse transcription/template switching nucleic acid primers described above. In some embodiments, the UMI sequence of each of the second nucleic acid primer described above in the plurality of nucleic acids of the kit is unique among the nucleic acids of the kit. In some embodiments, the plurality of nucleic acids comprises different populations of nucleic acid species. In certain embodiments, each population of nucleic acid species comprises a different barcode sequence that uniquely identifies a single population of nucleic acid species. In some embodiments, the kit further comprises a third nucleic acid primer comprising 12 to 32 nucleotides and a 5′ blocking group as described above. In some embodiments, the third nucleic acid is 22 nucleotides in length. An exemplary sequence of the third nucleic acid primer is 5′-ACACTCTTTCCCTACACGACGC-3′ (SEQ ID NO: 2). In some embodiments, the kit further comprises a nucleic acid comprising a barcode sequence. In some embodiments, the kit further comprises a phosphorothioate bond-containing nucleic acid comprising an X1*X2*X3*X4*X5*3′ sequence, wherein * is a phosphorothioate bond. In certain embodiments, the phosphorothioate bond-containing nucleic acid is 48 to 68 nucleotides in length, for example, 58 nucleotides in length. An exemplary sequence of the phosphorothioate bond-containing nucleic acid is 5′-AATGATACGGCGACCACCGAGATCTACACTCTTTCCCTACACGACGCTC TTCCG*A*T*C*T*3′ (SEQ ID NO: 3). In further embodiments, the kit further comprises a capture plate and/or a reverse transcriptase enzyme and/or a DNA purification column (e.g., a DNA purification spin column) and/or proteinase K.

For example, the kit can comprise a Moloney Murine Leukemia Virus (MMLV) reverse transcriptase, for example, SMARTscribe™ reverse transcriptase, SuperScript II™ reverse transcriptase, or Maxima H Minus™ reverse transcriptase. Exemplary kits include any one or any combinations of the reagents described herein and, optionally, directions for use. When multiple reagents and/or nucleic acids are provided in a single kit, the reagents may be provided in separate containers, such as separate tubes or vials. Optionally, the kit contains sterile water for use.

### Research Applications

In some embodiments, the nucleic acids, kits, and/or methods of the invention are used for research applications requiring sequencing or gene expression profiling. In certain embodiments, the research applications include studying cellular differentiation, characterizing tissue heterogeneity, high-throughput screening of agents (e.g., potential therapeutics, potential differentiation inducers, potential toxins, or any other agents whose effects on cells are of interest), stem cell reprogramming, cell lineage tracing, and virus detection in blood samples. Exemplary applications of the technology to the research context and proof are provided in the Examples and are merely illustrative of uses of the technology.

In certain embodiments, the nucleic acids (e.g., compositions), kits, and/or methods, of the disclosure are applied to gene expression analysis of single cells, optionally in response to contacting the single cell with an agent in the high-throughput screening context. The ability to analyze gene expression accurately and across large numbers of cells, and to be able to accurately correlate the expression level to a particular cell/well is an exemplary advantage and application of the instant technology. The technology is, in certain embodiments, similarly applied to other samples, such as cell or tissue lysates.

### Diagnosis, Prognosis, and Treatment

As described above, the invention is useful in generating a gene expression profile for a plurality of cells. These gene expression profiles can be used in a number of applications related to the diagnosis, prognosis, and treatment of a subject. For example, cells from a tissue sample collected from a patient can be used in the methods of the invention to generate an expression profile that can be compared against a known profile that is indicative of the disease or condition, thus informing a physician of whether the subject has the disease or condition. Similarly, the profile can be compared to a known profile useful in the prognosis of the disease or condition. For example, if the known profile is predictive of a cancer prognosis, the comparison may inform the physician of the stage of cancer or the cancer's likelihood of metastasis. In some embodiments, the invention can be used in a method of treating a disease or condition in a subject in need thereof. For example, a method of the invention can be used to obtain gene expression profiles in a subject before and after treatment with a therapeutic agent, thereby providing a means of determining the efficacy of the therapeutic agent. These data can be used to determine the efficacy of a treatment, or to help a physician determine an effective treatment regimen.

The invention is applicable to various diseases or conditions. Exemplary diseases or conditions are a cancer, a cardiovascular disease or condition, a neurological or neuropsychiatric disease or condition, an infectious disease or condition, a respiratory or gastrointestinal tract disease or condition, a reproductive disease or condition, a renal disease or condition, a prenatal or pregnancy-related disease or condition, an autoimmune or immune-related disease or condition, a pediatric disease, disorder, or condition, a mitochondrial disorder, an ophthalmic disease or condition, a musculo-skeletal disease or condition, or a dermal disease or condition.

All publications, patents and published patent applications referred to in this application are specifically incorporated by reference herein. In case of conflict, the present specification, including its specific definitions, will control.

Each embodiment described herein may be combined with any other embodiment described herein.

The following examples are provided to illustrate certain embodiments of the invention and are not intended to limit the scope of the invention.

### EXAMPLES

### Example 1

### Protocol for Transcriptome-Wide Single-Cell RNA Sequencing

To test the methods of the invention, the protocol described below was developed.

### Capture Plate Preparation

5 μL of lysis buffer, composed of a 1/500 dilution of Phusion HF buffer (New England Biolabs, #B0518S) were distributed in each well of a Twin.tec PCR 384-well collection plates (Eppendorf, #951020729).

### Cell Preparation

Media was removed by pelleting the cells for 5 min at 1000 rpm, and the RNA was immediately stabilized by resuspending the cells in 500 μL of RNAprotect Cell Reagent (Qiagen, #76526) and 1 μL of RNaseOUT Recombinant Ribonuclease Inhibitor (Life Technologies, #10777-019). Cells were stored up to two weeks at 4° C. Prior to sorting, cells in the RNAprotect Cell Reagent were diluted in 1.5 mL PBS, pH 7.4 (no calcium, no magnesium, no phenol red, Life Technologies, #10010-049). The cells then were stained for viability (DNA staining by Hoechst 33342) with NucBlue Live ReadyProbes Reagent (Life Technologies, #R37605).

### Cell Collection

Cells were sorted individually in each well of a 384-well capture plate using the FACSAria II flow cytometer (BD Biosciences). “Live” cells were selected and duplets avoided using the Hoechst DNA staining. In other words, following Hoechst staining, dead cells could be removed and not processed further and presence of a single cell/well could be confirmed. After sorting, the plates were immediately sealed, spun down, and frozen on dry ice. The sorted cells were stored at −80° C.

### Cell Lysis

Cells were thawed for 5 minutes at room temperature, then placed on ice.

### Reverse Transcription/Template Switching

1 μL of a 1×10⁻⁷ dilution of ERCC RNA Spike-In Mix (Life Technologies, #4456740) was added to each well. 1 μL of a universal adapter DNA primer (template-switching oligonucleotide) 5′-iCiGiCACACTCTTTCCCTACACGACGCrGrGrG-3′ (1 μM) (SEQ ID NO: 17) was added to each well, wherein iC represesents isocytosine (iso-dC), iG represents isoguanosine, and rG represents RNA guanosine. 1 μL of a cDNA synthesis primer 5′-/5Biosg/ACACTCTTTCCCTACACGACGCTCTTCCGATCT[BC6] N NNNTTTTTTTTTTTTTTTTTTTTTTTTTTTTTTVN-3′ (SEQ ID NO: 18) (1 μM) is added to each well, wherein SBiosg represents 5′ biotin, V represents a nucleotide selected from A, G, and C, N represents a nucleotide selected from A, G, C, and T, [BC6] represents a 6 base pair barcode sequence, different for each well of a 384 well plate, and (N)10 represents a Unique Molecular Identifier (UMI) sequence. The barcode sequences were designed such that each barcode differed from the others by at least two nucleotides, so that a single sequencing error could not lead to the misidentification of the barcode (Table 1). The plate was subsequently incubated at 72° C. for 3 minutes then immediately placed on ice to cool down (although this step is optional). The Template Switching step was carried out in each well using the following reagents: 2 μL of 5×1st strand buffer (250 mM UltraPure Tris-HCl, pH 8.0, Life Technologies, #15568-025; 375 mM KCl, LifeTechnologies, #AM9640G; 30 mM MgCl2, Life Technologies, #AM9530G); 1 μL of DL-Dithiothreitol solution BioUltra, 20 mM (Sigma-Aldrich, #43816); 1 μL of dNTPs (New England Biolabs, #N0447L); 0.254 of a MMLV Reverse Transcriptase, in this particular example, the MMLV reverse transcriptase SmartScribe Reverse Transcriptase (Clontech, #639538); and 0.754 of Nuclease-Free Water (not DEPC-Treated) water (LifeTechnologies, #AM9937). The plate was incubated at 42° C. for 1 hour 30 minutes.

TABLE 1
Exemplary bar code sequences

| Bar code | |
|---|---|
| sequence | Seq ID No. |
| AAAACT | 20 |
| AAAATC | 21 |
| AAACAT | 22 |
| AAACTA | 23 |
| AAAGTT | 24 |
| AAATAC | 25 |
| AAATCA | 26 |
| AAATGT | 27 |
| AAATTG | 28 |
| AACAAT | 29 |
| AACATA | 30 |
| AACTAA | 31 |
| AAGATT | 32 |
| AAGTAT | 33 |
| AAGTTA | 34 |
| AATAAC | 35 |
| AATACA | 36 |
| AATAGT | 37 |
| AATATG | 38 |
| AATCAA | 39 |
| AATCTT | 40 |
| AATGAT | 41 |
| AATGTA | 42 |
| AATTAG | 43 |
| AATTCT | 44 |
| AATTGA | 45 |
| AATTTC | 46 |
| ACAAAT | 47 |
| ACAATA | 48 |
| ACATAA | 49 |
| ACTAAA | 50 |
| ACTATT | 51 |
| ACTTAT | 52 |
| ACTTTA | 53 |
| AGAATT | 54 |
| AGATAT | 55 |
| AGATTA | 56 |
| AGTAAT | 57 |
| AGTATA | 58 |
| AGTTAA | 59 |
| ATAAAC | 60 |
| ATAACA | 61 |
| ATAAGT | 62 |
| ATAATG | 63 |
| ATACAA | 64 |
| ATACTT | 65 |
| ATAGAT | 66 |
| ATAGTA | 67 |
| ATATAG | 68 |
| ATATCT | 69 |
| ATATGA | 70 |
| ATATTC | 71 |
| ATCAAA | 72 |
| ATCATT | 73 |
| ATCTAT | 74 |
| ATCTTA | 75 |
| ATGAAT | 76 |
| ATGATA | 77 |
| ATGTAA | 78 |
| ATTAAG | 79 |
| ATTACT | 80 |
| ATTAGA | 81 |
| ATTATC | 82 |
| ATTCAT | 83 |
| ATTCTA | 84 |
| ATTGAA | 85 |
| ATTGTT | 86 |
| ATTTAC | 87 |
| ATTTCA | 88 |
| ATTTGT | 89 |
| ATTTTG | 90 |
| CAAAAT | 91 |
| CAAATA | 92 |
| CAATAA | 93 |
| CATAAA | 94 |
| CATATT | 95 |
| CATTAT | 96 |
| CATTTA | 97 |
| CTAAAA | 98 |
| CTAATT | 99 |
| CTATAT | 100 |
| CTATTA | 101 |
| CTTAAT | 102 |
| CTTATA | 103 |
| CTTTAA | 104 |
| GAAATT | 105 |
| GAATAT | 106 |
| GAATTA | 107 |
| GATAAT | 108 |
| GATATA | 109 |
| GATTAA | 110 |
| GTAAAT | 111 |
| GTAATA | 112 |
| GTATAA | 113 |
| GTTAAA | 114 |
| GTTATT | 115 |
| GTTTAT | 116 |
| GTTTTA | 117 |
| TAAAAC | 118 |
| TAAACA | 119 |
| TAAAGT | 120 |
| TAAATG | 121 |
| TAACAA | 122 |
| TAACTT | 123 |
| TAAGAT | 124 |
| TAAGTA | 125 |
| TAATAG | 126 |
| TAATCT | 127 |
| TAATGA | 128 |
| TAATTC | 129 |
| TACAAA | 130 |
| TACATT | 131 |
| TACTAT | 132 |
| TACTTA | 133 |
| TAGAAT | 134 |
| TAGATA | 135 |
| TAGTAA | 136 |
| TAGTTT | 137 |
| TATAAG | 138 |
| TATACT | 139 |
| TATAGA | 140 |
| TATATC | 141 |
| TATCAT | 142 |
| TATCTA | 143 |
| TATGAA | 144 |
| TATGTT | 145 |
| TATTAC | 146 |
| TATTCA | 147 |
| TATTGT | 148 |
| TATTTG | 149 |
| TCAAAA | 150 |
| TCAATT | 151 |
| TCATAT | 152 |
| TCATTA | 153 |
| TCTAAT | 154 |
| TCTATA | 155 |
| TCTTAA | 156 |
| TGAAAT | 157 |
| TGAATA | 158 |
| TGATAA | 159 |
| TGATTT | 160 |
| TGTAAA | 161 |
| TGTATT | 162 |
| TGTTAT | 163 |
| TGTTTA | 164 |
| TTAAAG | 165 |
| TTAACT | 166 |
| TTAAGA | 167 |
| TTAATC | 168 |
| TTACAT | 169 |
| TTACTA | 170 |
| TTAGAA | 171 |
| TTAGTT | 172 |
| TTATAC | 173 |
| TTATCA | 174 |
| TTATGT | 175 |
| TTATTG | 176 |
| TTCAAT | 177 |
| TTCATA | 178 |
| TTCTAA | 179 |
| TTGAAA | 180 |
| TTGATT | 181 |
| TTGTTA | 182 |
| TTTAAC | 183 |
| TTTACA | 184 |
| TTTAGT | 185 |
| TTTATG | 186 |
| TTTCAA | 187 |
| TTTCTT | 188 |
| TTTGTA | 189 |
| TTTTAG | 190 |
| TTTTCT | 191 |
| TTTTGA | 192 |
| TCTTTC | 193 |
| TTGGAT | 194 |
| ACCGTA | 195 |
| AGACCT | 196 |
| AGGGAT | 197 |
| ATCGAG | 198 |
| CAAGCT | 199 |
| CACCAA | 200 |
| CAGTCA | 201 |
| CATCAG | 202 |
| CATGGT | 203 |
| CCACAT | 204 |
| CCGATT | 205 |
| CGACTT | 206 |
| CGATTG | 207 |
| CTAGTG | 208 |
| CTTCTG | 209 |
| GAAGAC | 210 |
| GATCGT | 211 |
| GCTAGA | 212 |
| GCTTAC | 213 |
| GGACAT | 214 |
| GGCAAT | 215 |
| GGGATT | 216 |
| GTACAC | 217 |
| GTCAAG | 218 |
| GTGACT | 219 |
| GTTCGA | 220 |
| TAGTGG | 221 |
| TCCAAC | 222 |
| TCGAAG | 223 |
| TCTGCA | 224 |
| TTCCTC | 225 |
| TTGTCC | 226 |
| TTTGGC | 227 |
| CCAACC | 228 |
| CCTTCC | 229 |
| CTCTCC | 230 |
| GGACCA | 231 |
| GTACCG | 232 |
| ACCCCC | 233 |
| ACCCGG | 234 |
| ACCGCG | 235 |
| ACCGGC | 236 |
| ACGCCG | 237 |
| ACGCGC | 238 |
| ACGGCC | 239 |
| ACGGGG | 240 |
| AGCCCG | 241 |
| AGCCGC | 242 |
| AGCGCC | 243 |
| AGCGGG | 244 |
| AGGCCC | 245 |
| AGGCGG | 246 |
| AGGGCG | 247 |
| AGGGGC | 248 |
| CACCCC | 249 |
| CACCGG | 250 |
| CACGCG | 251 |
| CACGGC | 252 |
| CAGCCG | 253 |
| CAGCGC | 254 |
| CAGGCC | 255 |
| CAGGGG | 256 |
| CCACCG | 257 |
| CCACGC | 258 |
| CCAGGG | 259 |
| CCCACG | 260 |
| CCCAGC | 261 |
| CCCCAC | 262 |
| CCCCCA | 263 |
| CCCCGT | 264 |
| CCCCTG | 265 |
| CCCGAG | 266 |
| CCCGGA | 267 |
| CCCTGG | 268 |
| CCGAGG | 269 |
| CCGCAG | 270 |
| CCGCGA | 271 |
| CCGGAC | 272 |
| CCGGCA | 273 |
| CCGGGT | 274 |
| CCGGTG | 275 |
| CCGTCG | 276 |
| CCGTGC | 277 |
| CCTCGG | 278 |
| CCTGCG | 279 |
| CCTGGC | 280 |
| CGACCC | 281 |
| CGACGG | 282 |
| CGAGCG | 283 |
| CGAGGC | 284 |
| CGCACC | 285 |
| CGCAGG | 286 |
| CGCCAG | 287 |
| CGCCCT | 288 |
| CGCCGA | 289 |
| CGCCTC | 290 |
| CGCGAC | 291 |
| CGCGCA | 292 |
| CGCGGT | 293 |
| CGCGTG | 294 |
| CGCTCG | 295 |
| CGCTGC | 296 |
| CGGACG | 297 |
| CGGAGC | 298 |
| CGGCAC | 299 |
| CGGCCA | 300 |
| CGGCGT | 301 |
| CGGCTG | 302 |
| CGGGAG | 303 |
| CGGGCT | 304 |
| CGGGGA | 305 |
| CGGGTC | 306 |
| CGGTCC | 307 |
| CGGTGG | 308 |
| CGTCCG | 309 |
| CGTCGC | 310 |
| CGTGCC | 311 |
| CGTGGG | 312 |
| CTCCCG | 313 |
| CTCCGC | 314 |
| CTCGGG | 315 |
| CTGCGG | 316 |
| CTGGCG | 317 |
| CTGGGC | 318 |
| GACCCG | 319 |
| GACCGC | 320 |
| GACGCC | 321 |
| GACGGG | 322 |
| GAGCCC | 323 |
| GAGCGG | 324 |
| GAGGCG | 325 |
| GAGGGC | 326 |
| GCACCC | 327 |
| GCACGG | 328 |
| GCAGCG | 329 |
| GCAGGC | 330 |
| GCCACC | 331 |
| GCCAGG | 332 |
| GCCCAG | 333 |
| GCCCCT | 334 |
| GCCCGA | 335 |
| GCCCTC | 336 |
| GCCGAC | 337 |
| GCCGCA | 338 |
| GCCGGT | 339 |
| GCCGTG | 340 |
| GCCTCG | 341 |
| GCCTGC | 342 |
| GCGACG | 343 |
| GCGAGC | 344 |
| GCGCAC | 345 |
| GCGCCA | 346 |
| GCGCGT | 347 |
| GCGCTG | 348 |
| GCGGAG | 349 |
| GCGGCT | 350 |
| GCGGGA | 351 |
| GCGGTC | 352 |
| GCGTCC | 353 |
| GCGTGG | 354 |
| GCTCCG | 355 |
| GCTCGC | 356 |
| GCTGCC | 357 |
| GCTGGG | 358 |
| GGACGC | 359 |
| GGAGCC | 360 |
| GGAGGG | 361 |
| GGCACG | 362 |
| GGCAGC | 363 |
| GGCCAC | 364 |
| GGCGAG | 365 |
| GGCGCT | 366 |
| GGCGGA | 367 |
| GGCGTC | 368 |
| GGCTCC | 369 |
| GGGACC | 370 |
| GGGAGG | 371 |
| GGGCAG | 372 |
| GGGCCT | 373 |
| GGGCGA | 374 |
| GGGCTC | 375 |
| GGGGAC | 376 |
| GGGGCA | 377 |
| GGGGGT | 378 |
| GGGGTG | 379 |
| GGGTCG | 380 |
| GGGTGC | 381 |
| GGTCCC | 382 |
| GGTGCG | 383 |
| GGTGGC | 384 |
| GTCCCC | 385 |
| GTCGCG | 386 |
| GTCGGC | 387 |
| GTGCGC | 388 |
| GTGGCC | 389 |
| GTGGGG | 390 |
| TCCCCG | 391 |
| TCCCGC | 392 |
| TCCGGG | 393 |
| TCGCGG | 394 |
| TCGGCG | 395 |
| TCGGGC | 396 |
| TGCCCC | 397 |
| TGCGCG | 398 |
| TGCGGC | 399 |
| TGGCCG | 400 |
| TGGCGC | 401 |
| TGGGCC | 402 |
| TGGGGG | 403 |

cDNA Pooling and Purification

All 384 wells were pooled together, and 35 mL of DNA Binding Buffer (Zymo Research, #D4004-1-L) was added to the pooled cDNAs. All cDNAs pooled from one 384-well plate were purified through a DNA purification spin column, in this case, one single DNA Clean & Concentrator-5 column (Zymo Research, #D4013), and the cDNAs were eluted in 17 μL of Nuclease-Free Water.

### Exonuclease I Treatment

Pooled cDNAs were treated with an exonuclease, in this case Exonuclease I, 24 of 10× reaction buffer, 1 μL of Exonuclease I (New England Biolabs, #MO293L), and the reaction was incubated at 37° C. for 30 minutes, then at 80° C. for 20 minutes.

Full Length cDNA Amplification

Full length cDNA was amplified by single primer PCR using the Advantage 2 PCR Enzyme System (Clontech, #639206). The PCR reaction was set up as follows: 204 of cDNA from previous step; 54 of 10× Advantage 2 PCR buffer; 1 μL of dNTPs; 1 μL of the DNA primer 5′-/5Biosg/ACACTCTTTCCCTACACGACGC-3′ (SEQ ID NO: 19) (wherein 5Biosg represents 5′ biotin) (10 μM, Integrated DNA Technologies); 1 μL of the Advantage 2 Polymerase Mix; and 22 μL of Nuclease-Free Water, and performed using the following program: 95° C. for 1 minute; 18 cycles of a) 95° C. for 15 seconds, 65° C. for 30 seconds, 68° C. for 6 minutes, and 72° C. for 10 minutes (followed by an option hold period at 4° C.).

Full Length cDNA Purification and Quantification

Full length cDNAs were purified with 304 of beads (here, Agencourt AMPure XP magnetic beads (Beckman Coulter, #A63880)). The full length cDNAs were eluted in 124 of Nuclease-Free Water and quantified on the Qubit 2.0 Flurometer (Life Technologies) using the dsDNA HS Assay (Life Technologies #Q32851).

### Sequencing Library Preparation

From the purified full length cDNA, 1 ng of cDNA was engaged in Nextera library preparation according to the Illumina protocol, with the exception that in the Illumina protocol, only the i7 primer (e.g., a primer which is standard to the Illumina system) was used to barcode cDNA originating from the same 384-well plate, whereas we also use 5 μM of a second primer (5′-AATGATACGGCGACCACCGAGATCTACACTCTTTCCCTACACGACGCTC TTCCG*A*T*C*T*-3′ (SEQ ID NO: 3), wherein * represents a phosphorothioate bond) during the library amplification step.

### Sequencing Library Purification and Size Selection

The resulting sequencing library was purified with 30 μL of Agencourt AMPure XP magnetic beads and eluted in 204 of nuclease free water. The entire library was run on an E-Gel EX Gel, 2% (Life Technologies, #G4010-02), and the band corresponding to a size range of 300 to 800 bp was excised and purified using the QIAquick Gel Extraction Kit (Qiagen, #28704).

### Sequencing Library Quality Assessment

The library was quantified on the Qubit 2.0 Fluorometer using the dsDNA HS Assay. The quality and average size of the library were assessed by BioAnalyzer (Agilent) with the High Sensitivity DNA kit (Agilent, #5067-4626).

### Sequencing

Sequencing is performed on any Illumina® HiSeg™ or MiSeg™ using standard Illumina® sequencing kit. Libraries are run on paired-end flow cells by running 17 cycles on the first strand, then 8 cycles to decode the Nextera™ barcode and finally 34 cycles (although 46 cycles also can be used to increase the amount of sequencing data). Up to twelve Nextera libraries/384-well capture plates, each comprising 384 cells, are multiplexed together (twelve libraries can be used with a set of twelve plate-identifying barcode sequences, although this number can be expanded with additional barcode sequences), allowing the simultaneous sequencing of up to 4,608 single cell transcriptomes on a single lane.

### Example 2

### Single Cell Sequencing of Differentiating Stem Cells

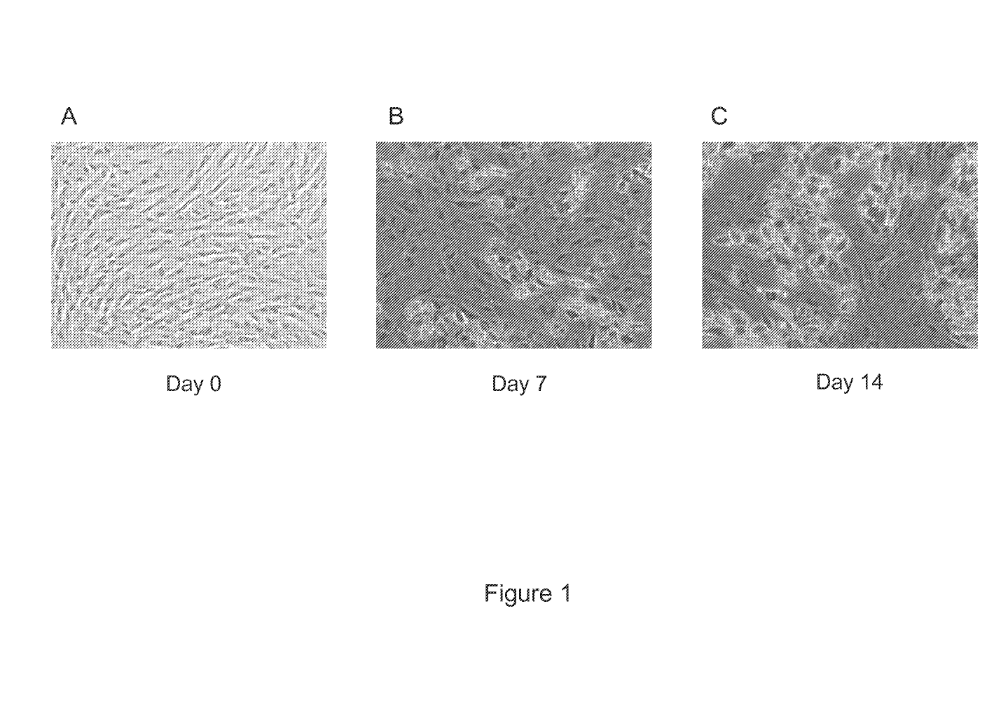

The methods and reagents (e.g., polynucleotides, kits, etc.) described herein have numerous applications. The following provides an example demonstrating the application of the instant technology to a particular context. The method described above was used to sequence the transcriptomes of a population of differentiating human adipose tissue-derived stromal/stem cells (hASCs) at three different time points (day 0, day 1, day 2, day 3, day 5, day 7, day 9, and day 14). Visual inspection of these cells indicates that differentiation over time is incomplete, thus leading to a heterogeneous cell population (FIG. 1). Given the heterogeneous appearance of the cells, we would expect that, if cells in the culture could be rigorously analyzed at the single cell level and gene expression accurately correlated with each specific single cell, expression of genes relevant to differentiation and other activities would differ across individual cells at a given time point. We thus undertook such analysis as proof of principle of the robustness of the methods and compositions of the present invention.

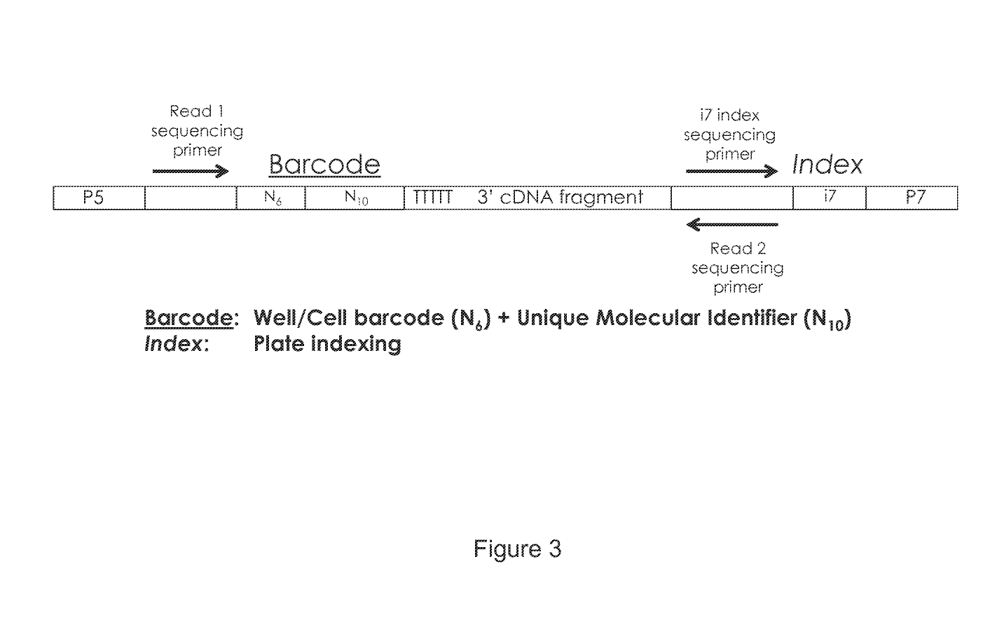

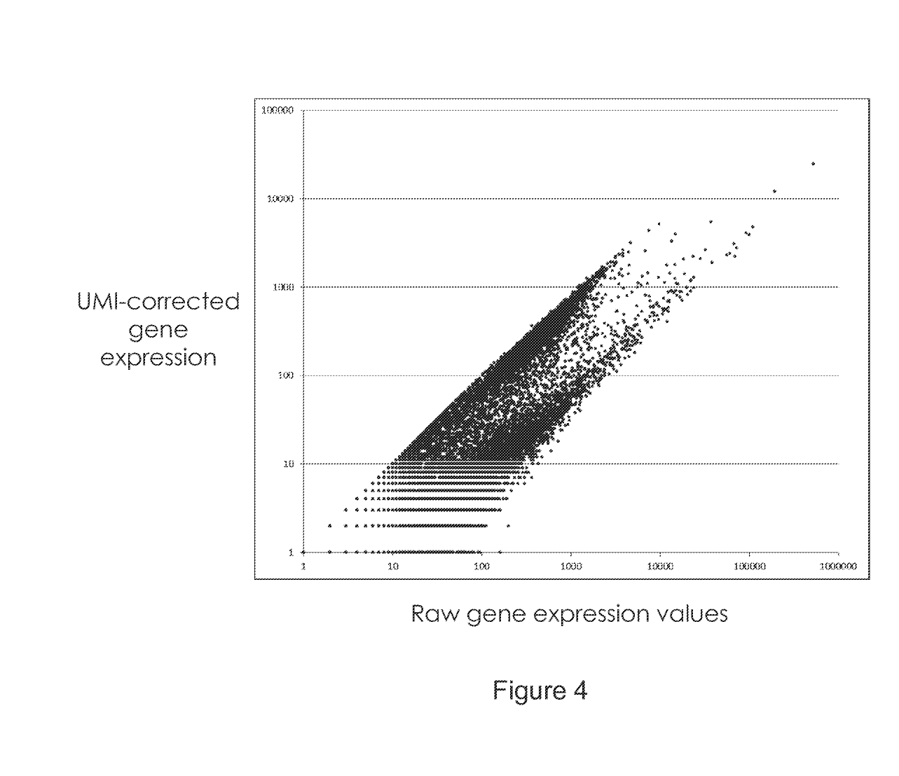

As proof of principle, single-cell RNA-seq data were generated for 9,216 cells in total that represent 1,152 cells collected for each of the eight time points profiled (day 0, day 1, day 2, day 3, day 5, day7, day 9, and day 14). To generate these data, FACS was used to sort the cells into 24 384-well plates. FIG. 3 depicts the design of the sequencing library incorporating the two levels of barcoding (well/cell and plate), the UMI, and the primer sequences indicated as P5 and P7 for Illumina sequencing. P5 and P7 are the regions that anneal to their complementary oligos on the flow cell. The index (i7) represents the plate index than is added during the Nextera tagmentation process after all wells have been pooled and pre-amplified. It is incorporated by PCR during the last step of the library preparation. One i7 index is used per pool/plate of 96 or 384 samples/cells, allowing for a higher level of multiplexing by pooling several plates together for sequencing. The sequencing primers P5 and P7 initiate the sequencing reaction. The sequencing will result in 3 distinct reads. The first one is 16 bp long and includes 6 bp of the well/cell barcode followed by 10 bp of the UMI. Then the i7 index sequencing primer allows us to read the plate/pool index (i7, 8 bp) on the same strand. Finally, the other strand is generated (paired-end sequencing) and the read 2 sequencing primer allows us to read the actual cDNA fragment, which is typically 45 bp with a 50 cycle kit. By using the 3 reads and deciphering the barcodes, we can trace each cDNA to a specific well, plate, and transcript. In certain embodiments, the disclosure provides a polynucleotide as set forth on FIG. 3 (e.g., a polynucleotide comprising various polynucleotide portions, such as contiguous portions, as set forth in FIG. 3). The various portions are described herein and the figure contemplates polynucleotides comprising any combinations of these various portion. Expression values were correlated by comparing raw read counts to UMI counts (FIG. 4). Incorporating and counting UMIs helped to reduce the PCR bias.

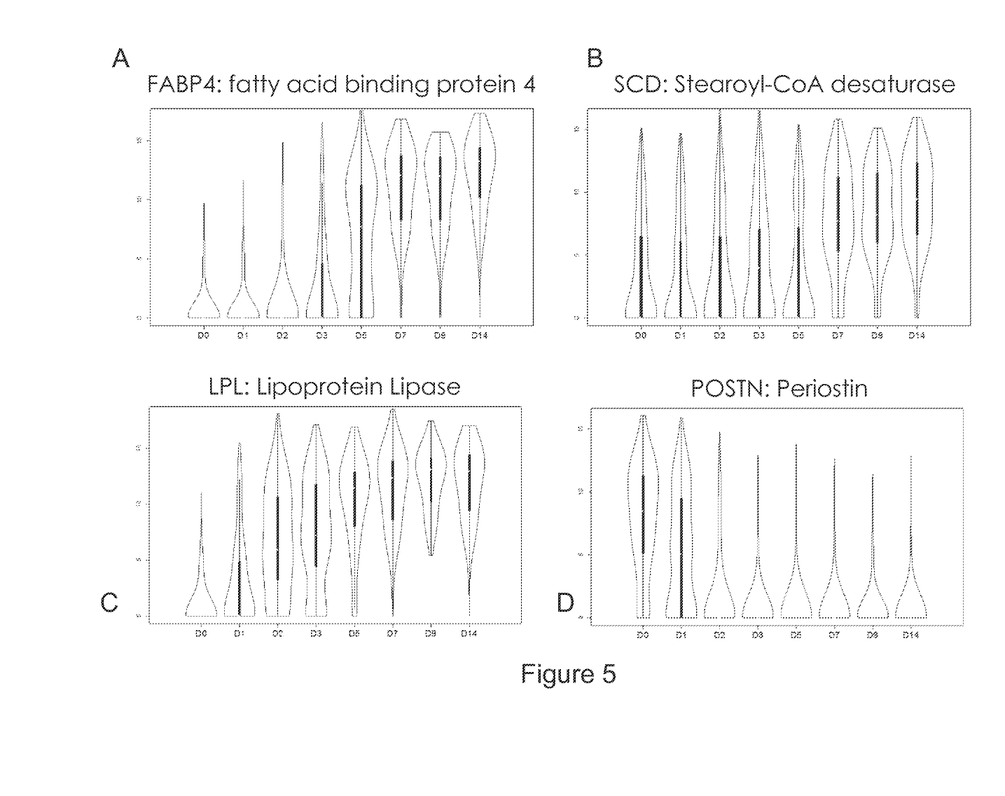

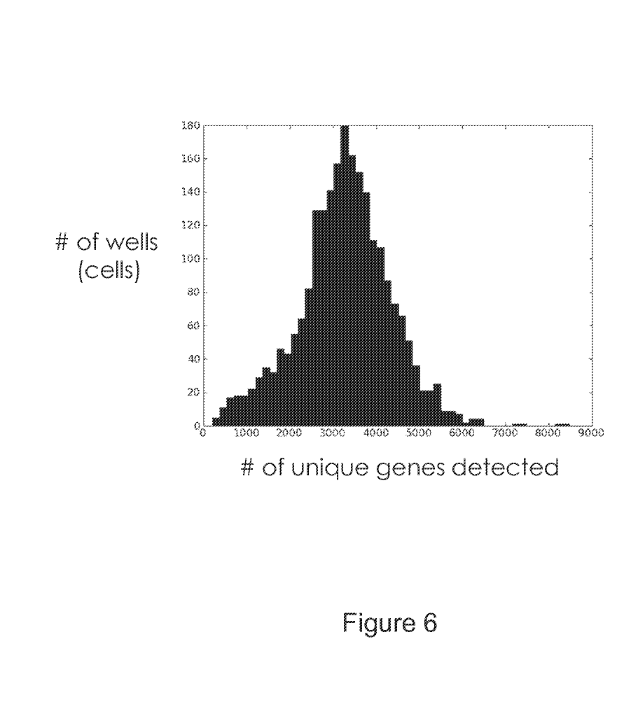

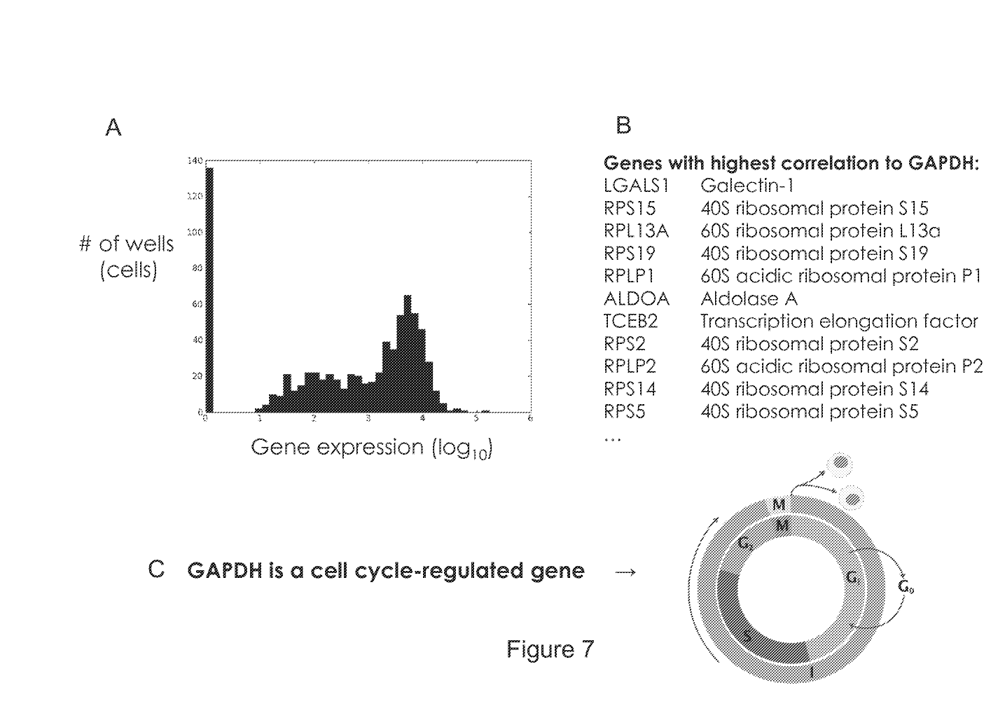

Key marker genes among the cells for each time point were measured, and the distribution of expression levels was plotted over time (days 0 to 14) as shown in FIG. 5. With the single cell RNA-seq data, the proportions of cells expressing a gene at a given level are observable. Gene detection in single cells was plotted as a histogram showing how many expressed genes were detected per cell (FIG. 6). By way of exemplifying the data for a gene, GAPDH was selected as an example of a “housekeeping” gene that shows a burst of transcription and that is a cell cycle-regulated gene. The histogram of FIG. 7 represents the distribution of GAPDH expression among the cells profiled at day 0. While GAPDH usually is present at a constant level of expression in a population of cells, when observed at the single cell level, a significant portion of cells were seen that did not express GAPDH because GAPDH is a cell cycle-regulated gene. Thus, by using the single cell sequencing method, we revealed that, despite its widespread use as a “housekeeping” reference gene, GAPDH is not necessarily a good reference gene especially at the single cell level. This underscores the power of the single cell sequencing methods of the invention.

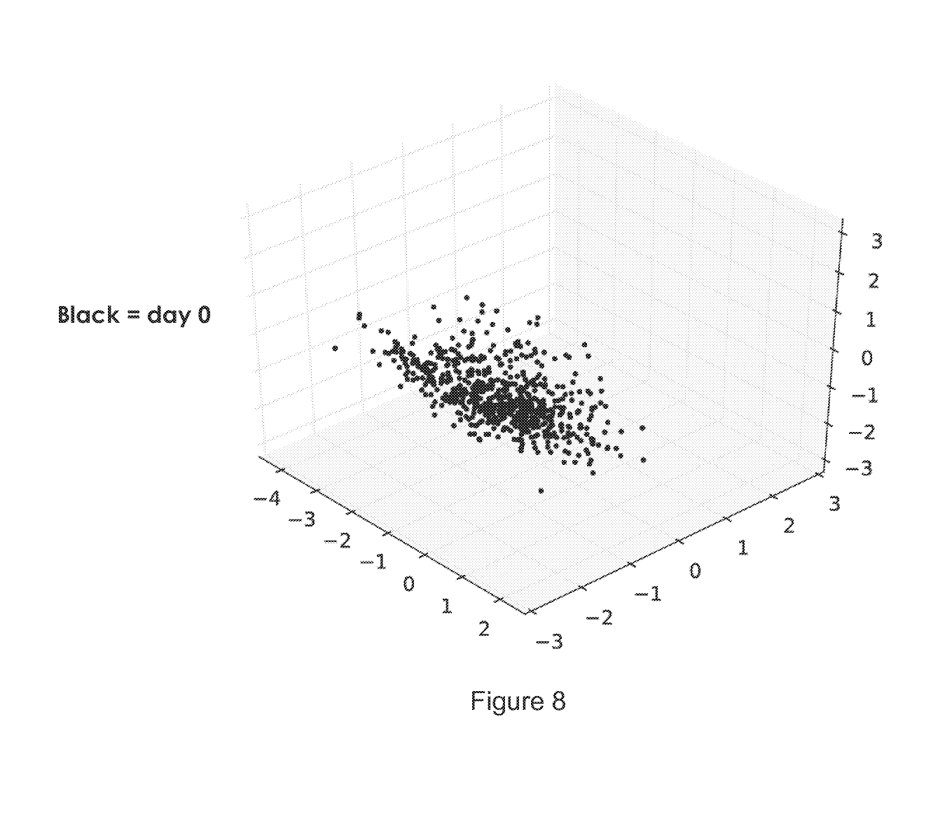

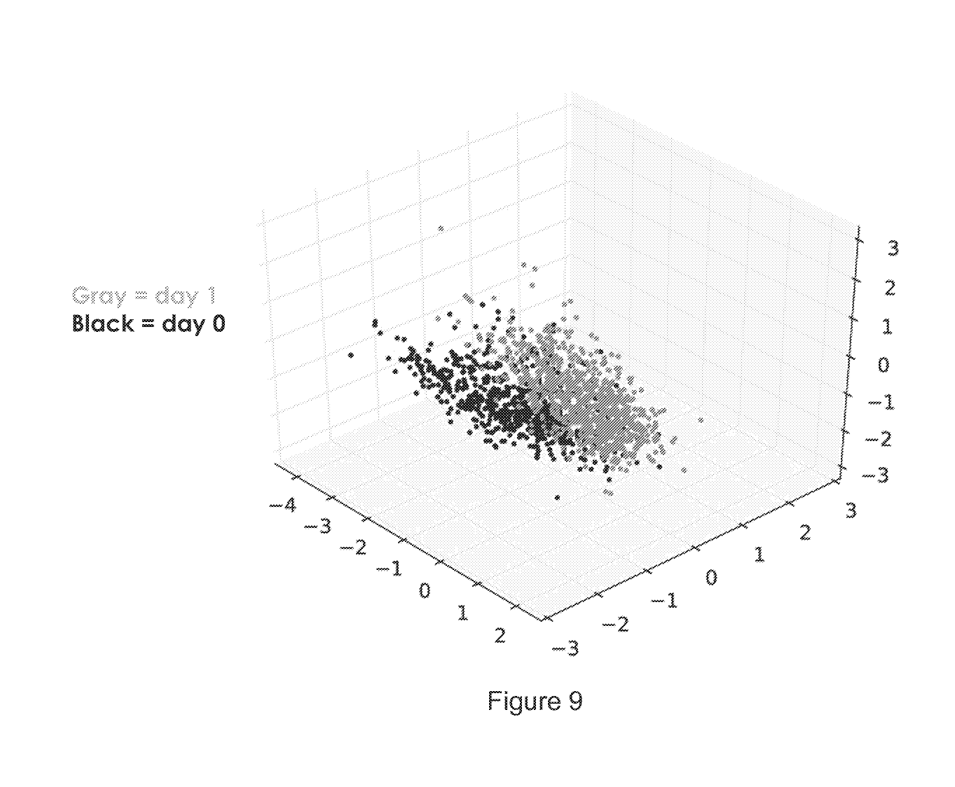

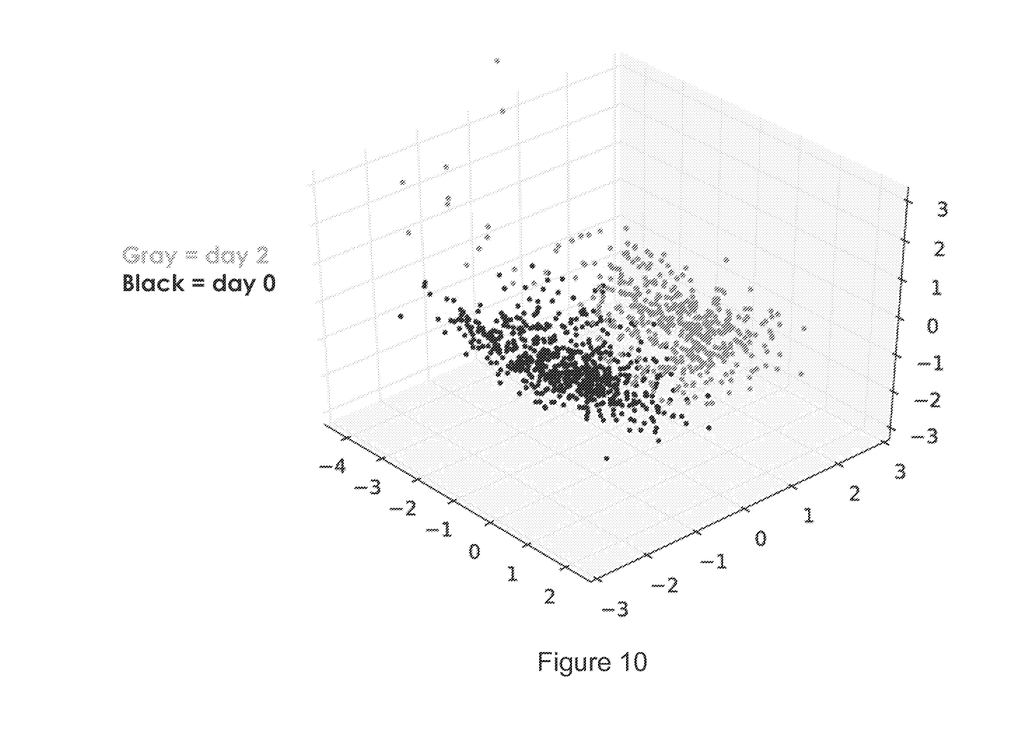

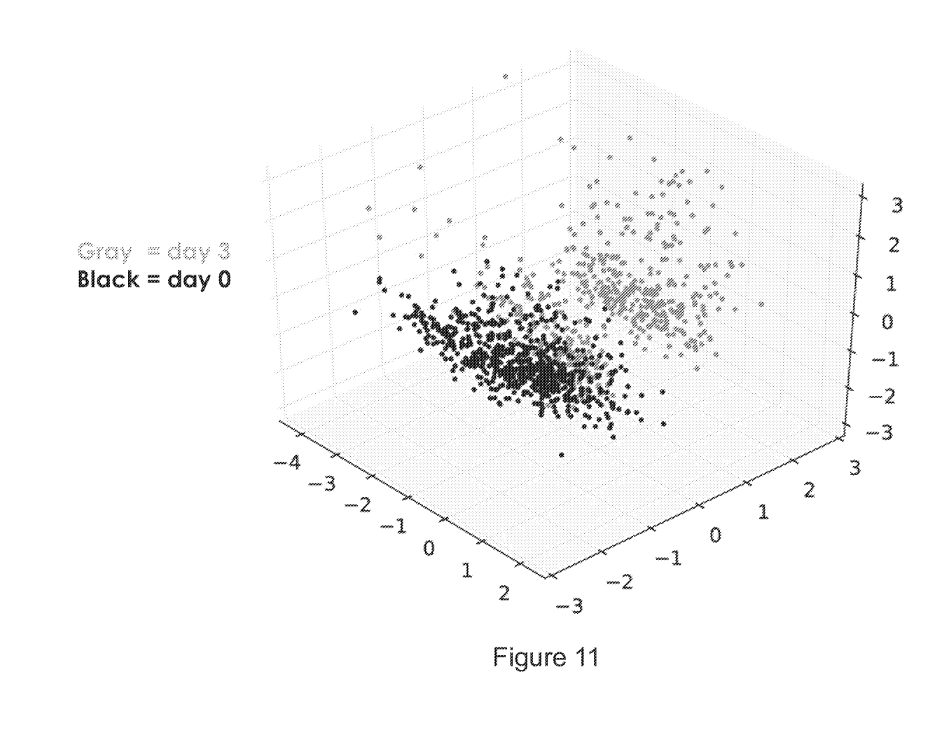

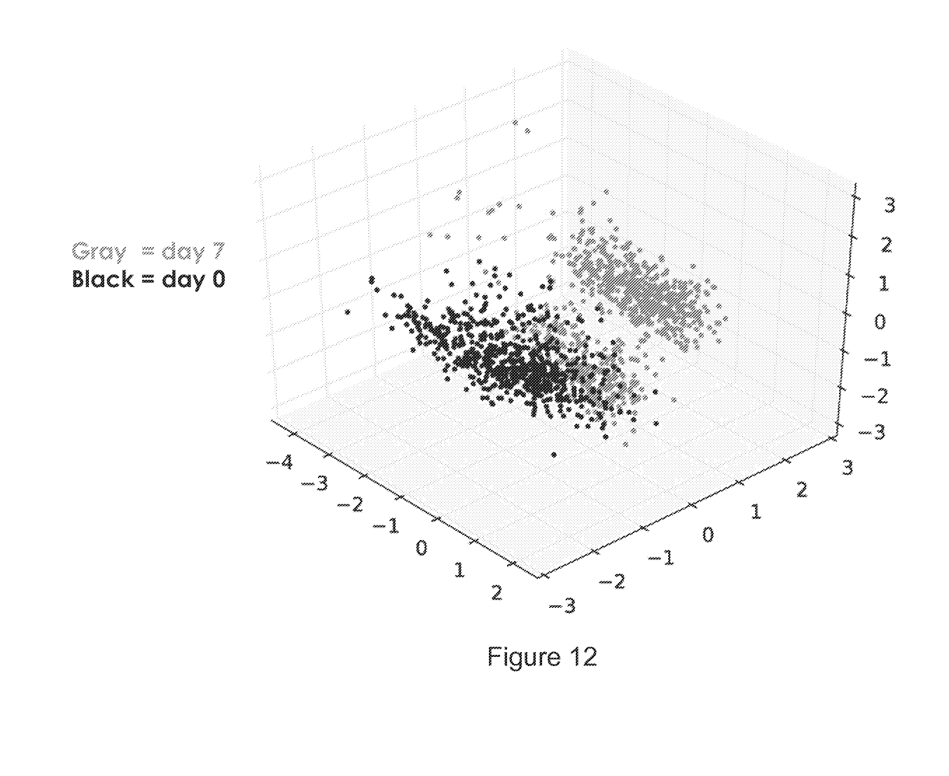

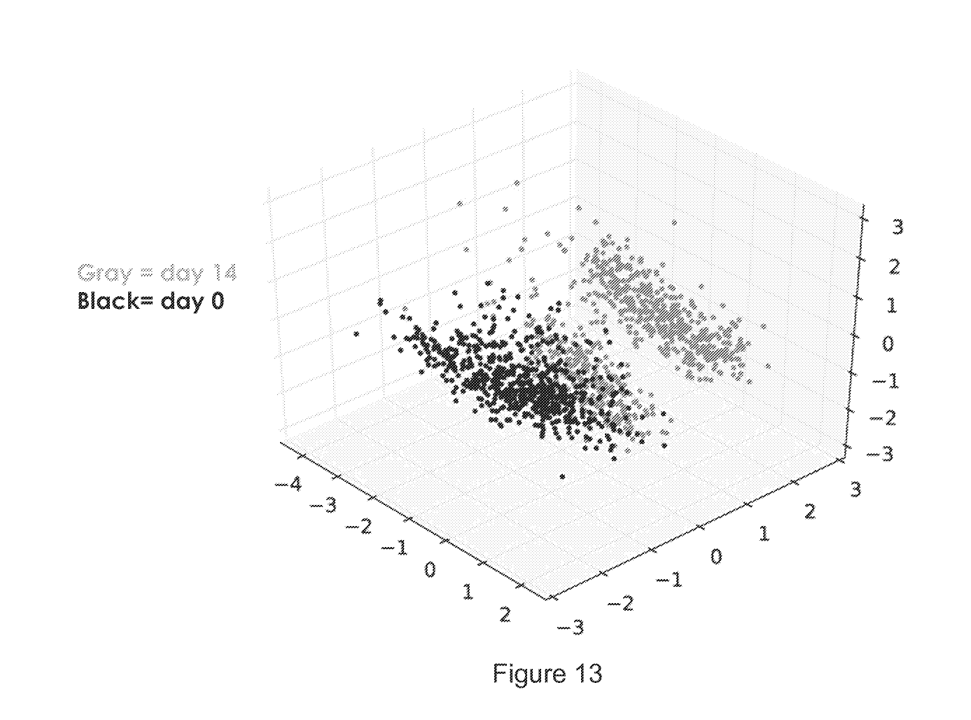

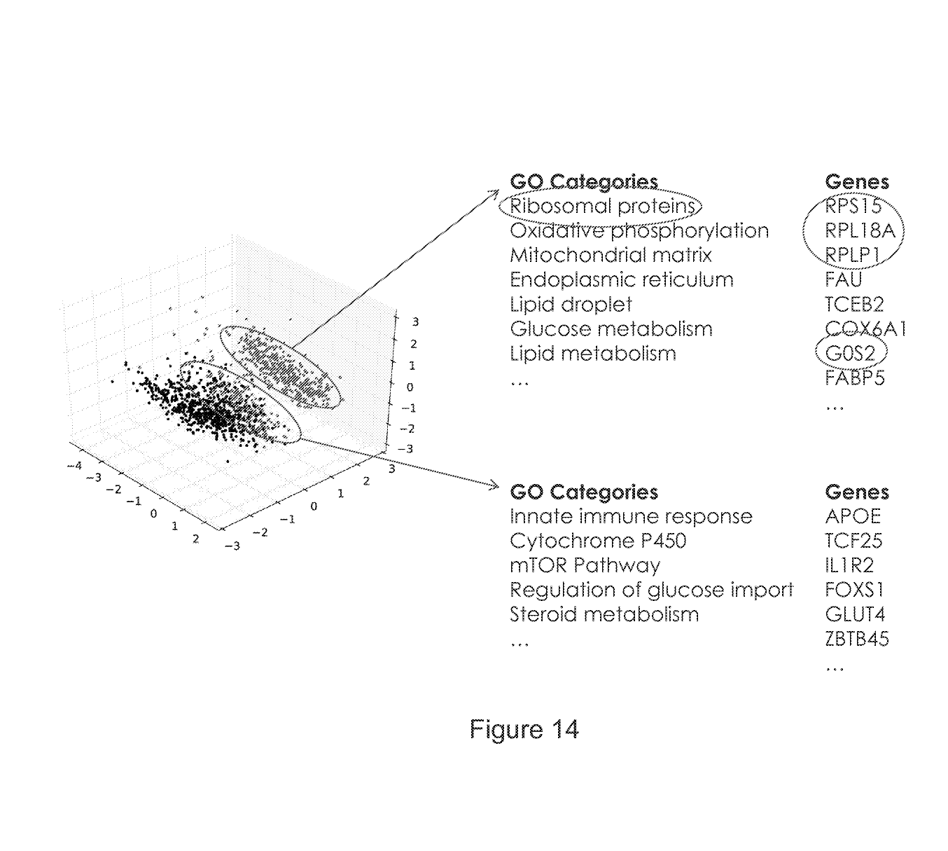

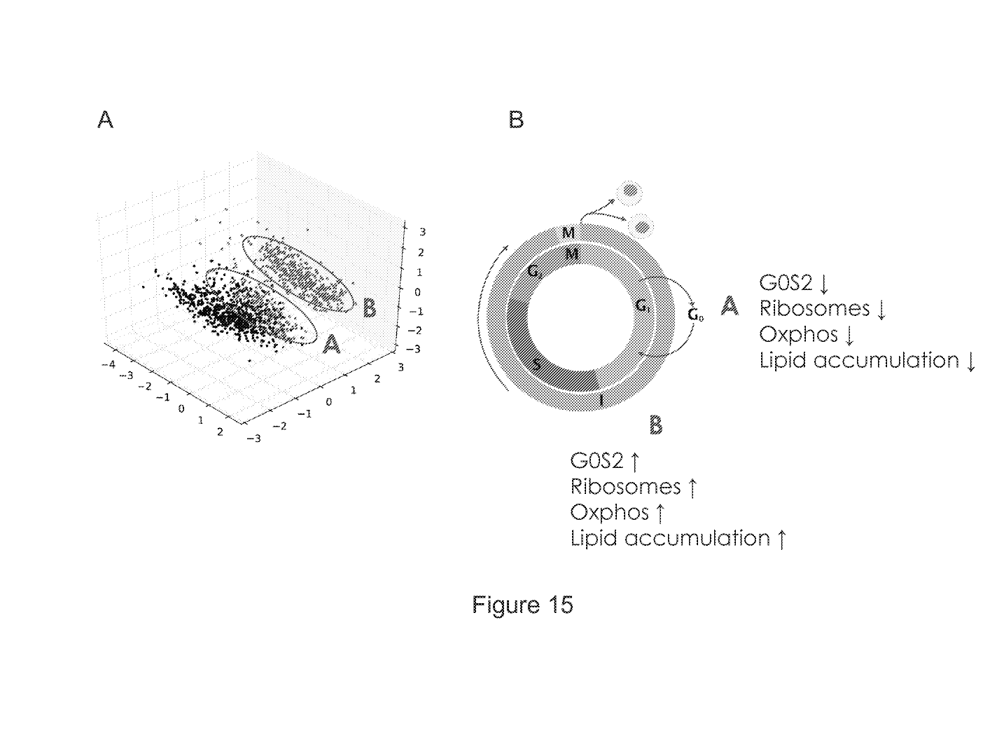

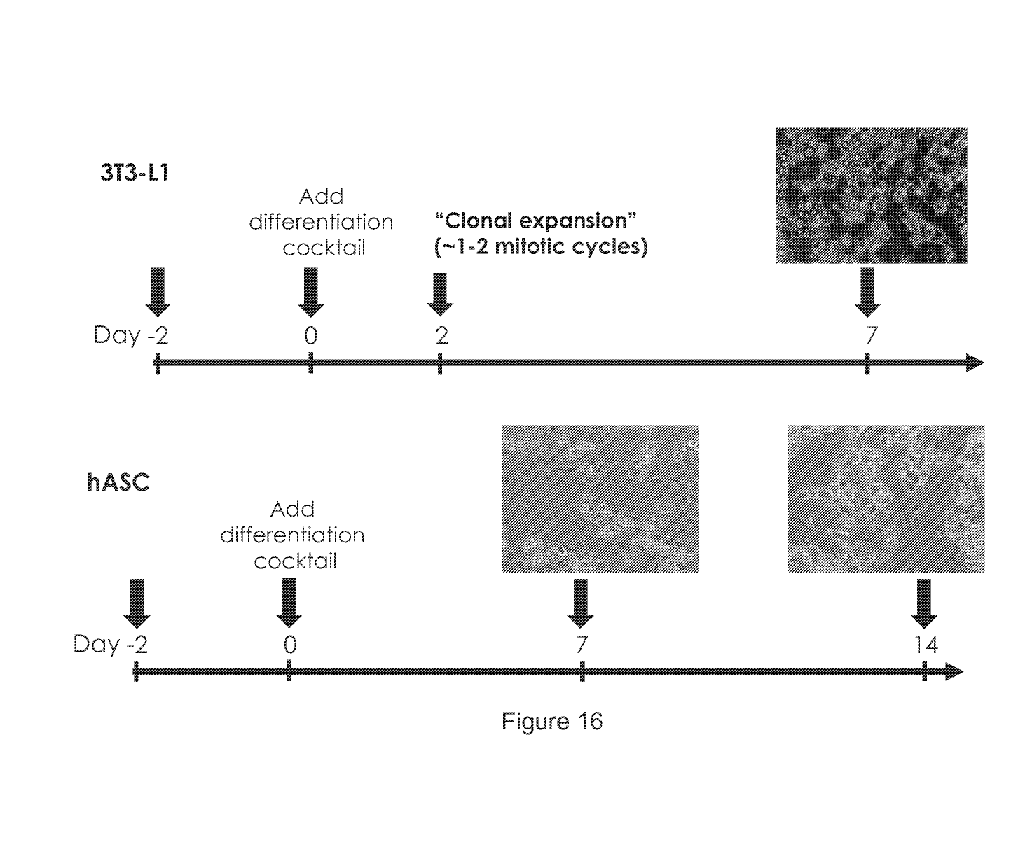

A projection of three of the highest components of a principal component analysis based on gene expression are shown in FIGS. 8 to 13. Each point represents a profiled cell. The cells profiled at day 0 are represented in black, while the cells profiled at the subsequent time points (day 1, day 2, day 3, day 7, and day 14) are shown in gray (or in red if depicted in color). A clear distinction can be seen between the day 0 cells and the cells from subsequent time points. To explore these differences, a Gene Ontology analysis then was performed on the differentially expressed genes between two subpopulations distinguishable at day 14 with the principal component analysis: a subpopulation of genes that clusters with day 0 genes and a subpopulation that is separate from those genes. Key genes that characterize these two day 14 subpopulations were identified and categorized using the Gene Ontology database (FIG. 14). The ability to distinguish these subpopulations illustrates the robustness of the methodology. A partial conclusion of these analyses shows the link between the expression of adipocyte genes and G-1 arrest (FIG. 15). Based on this analysis, it appears that one subpopulation fully differentiates, while the other seems to be stuck in the G0 phase and cannot fully differentiate. These data were then further used in a comparison of adipogenesis efficiency between a mouse system (3T3-L1) where the differentiation process is much more efficient and for which there is a clonal expansion, and in human cells (hASCs), where this clonal expansion is absent (FIG. 16). This clonal expansion may be essential to avoid a subpopulation becoming stuck in the G0 phase and resulting in incomplete differentiation.

In conclusion, the data show that the invention provides a useful method for single cell sequencing and single transcript tracking that uses the aggregation of samples and subsequent deconvolution of data. Through this process of aggregation and deconvolution, the sequencing can be performed with less cost and greater efficiency than by traditional sequencing techniques. Moreover, the results obtained here reflect the ability to detect changes and differences across heterogeneous populations when those populations are evaluated at the single cell level. Such changes and differences may be lost (e.g., averaged out) if gene expression across the heterogenous population is instead evaluated.

### Example 3

### Simultaneous Single Cell Sequencing of 12,832 Cells

To further demonstrate the applicability of single cell sequencing methods and compositions (e.g., reagents, nucleic acids, kits) of the disclosure for addressing a range of questions, including questions related to understanding cell and developmental biology, a primary human adipose-derived stem/stromal cell (hASC) differentiation system was used as a test system, akin to that described above. Once again, single cell RNA sequencing methods and compositions of the invention was successfully used to survey gene expression in differentiating hASC cultures at single cell resolution. The resulting data reveal the major axes of variation on gene expression, suggest a biological basis for the morphological heterogeneity observed in these cultures, and provide a rich resource for dissection of the regulatory networks involved in adipocyte formation and function beyond what investigations using other techniques have shown. Through advances in sequencing and cell isolation technologies, identification of rare expression programs can be enabled by deeper and more sensitive profiling of every cell, and direct comparison of in vitro and in vivo heterogeneity can be observed through direct profiling of single cells from tissue samples.

The protocol used in this particular example was as follows.

### Cell Culture

Human adipose-derived stem/stromal cells (hASCs) were isolated from lipoaspirates and purified by flow-cytometry (CD29, CD44, CD73, CD90, CD105 and CD166 positive; CD14, CD31, CD45 and Lin1 negative) (cells were obtained from Life Technologies). The hASCs were cultured in a 2% reduced serum medium (MesenPro RS, Life Technologies) and expanded for no more than 3 passages. The cultures were then induced to differentiate towards an adipogenic fate after reaching 80% confluency (differentiations D1 and D2) or two days after reaching 100% confluency (differentiation D3) by switching from growth medium to the StemPro adipogenesis differentiation medium (Life Technologies), and were subsequently prepared for further analysis, such as by qPCR or smFISH. Following induction, the differentiation medium was changed every three days for up to 14 days. The variation in initial conditions (confluency upon differentiation) was introduced to assess the robustness of the subsequent time course data.

### Single Cell Isolation

Cells were harvested using TrypLE Express (Life Technologies) and medium removed by pelleting the cells in a centrifuge (5 minutes at 1000 rpm). RNA was stabilized by immediately resuspending the pelleted cells in RNAprotect Cell Reagent (Qiagen) and RNaseOUT Recombinant Ribonuclease Inhibitor (Life Technologies) at a 1:1000 dilution. Just prior to fluorescence-activated cell sorting (FACS), the cells were diluted in PBS (pH 7.4, no calcium, magnesium or phenol red; Life Technologies) and stained for viability using Hoechst 33342 (Life Technologies). 384-well SBS capture plates were filled with 5 μl of a 1:500 dilution of Phusion HF buffer (New England Biolabs) in water and cells were then sorted into each well using a FACSAria II flow cytometer (BD Biosciences) based on Hoechst DNA staining After sorting, the plates were immediately sealed, spun down, cooled on dry ice, and stored at −80° C. For lipid content-based FACS, cells were also stained with HSC LipidTOX Neutral Lipid Stain (Life Technologies) and sorted according to their relatively “high” or “low” lipid content, either by taking the top and bottom 20% of stained cells (D2) or the top and bottom 50% (D3).

### Sequencing of Sorted Single Cells

Frozen cells were thawed for 5 minutes at room temperature. For the second time course (D3) only, lysis conditions further included treating the cells with proteinase K (200 μg/mL; Ambion), followed by RNA desiccation to inactivate the proteinase K and simultaneously reduce the reaction volume. The cells were kept at 50° C. for 15 minutes in a sealed plate, then 95° C. for 10 minutes with the seal removed.

### Primers

The primers used, and the resulting products, are as follows.

1st Strand cDNA

5′-RNA:NB(A)30-3′
3′-
CCC:cDNA:NV(T)30(N)10[BC6]TCTAGCCTTCTCGCAGCACATCC
CTTTCTCACA-5′

2nd Strand cDNA

5′-ACACTCTTTCCCTACACGACGCGGG:cDNA:NB(A)30-3′
CCC:cDNA:NV(T)30(N)10[BC6]TCTAGCCTTCTCGCAGCACATCCC
TTTCTCACA-5′

Resulting Full Length cDNA

5′-
ACACTCTTTCCCTACACGACGCGGG:cDNA:NB(A)30(N)10[BC6]AG
ATCGGAAGAGCGTCGTGTAGGGAAAGAGTGT-3′
3′-
TGTGAGAAAGGGATGTGCTGCGCCC:cDNA:NV(T)30(N)10[BC6]TC
TAGCCTTCTCGCAGCACATCCCTTTCTCACA-5′

Full Length cDNA Amplification:

### Single Primer PCR

3-′CGCAGCACATCCCTTTCTCACA-5′
5′-
ACACTCTTTCCCTACACGACGCGGG:cDNA:NB(A)30(N)10[BC6]AG
ATCGGAAGAGCGTCGTGTAGGGAAAGAGTGT-3′
3,-
TGTGAGAAAGGGATGTGCTGCGCCC:cDNA:NV(T)30(N)10[BC6]TC
TAGCCTTCTCGCAGCACATCCCTTTCTCACA-5′
5′-ACACTCTTTCCCTACACGACGC-3′

### Transposon Based Library (Nextera)

### Tagmentation

5′-ACACTCTTTCCCTACACGACGCTCTTCCGATCT[BC6](N)10(T)
30VN-Frag-3′
3′-Frag-GACAGAGAATATGTGTAGAGGCTCGGGTGCTCTG-5′

### Library Amplification (Modified)

3′-GGCTCGGGTGCTCTG[i7]TAGAGCATACGGCAGAAGACGAAC-5′
5′-ACACTCTTTCCCTACACGACGCTCTTCCGATCT[BC6](N)10(T)
30VN-Frag-CTGTCTCTTATACACATCTCCGAGCCCACGAGAC-3′
3′-TGTGAGAAAGGGATGTGCTGCGAGAAGGCTAGA[BC6](N)10(A)
30BN-Frag-GACAGAGAATATGTGTAGAGGCTCGGGTGCTCTG-5′
5′-
AATGATACGGCGACCACCGAGATCTACACTCTTTCCCTACACGACGCTC
TTCCGATCT-3′

### Resulting Library

5′-AATGATACGGCGACCACCGAGATCTACACTCTTTCCCTACACGAC
GCTCTTCCGATCT[BC6](N)10(T)30VN-Frag-CTGTCTCTTAT
ACACATCTCCGAGCCCACGAGAC[i7]ATCTCGTATGCCGTCTTCTGC
TTG-3′
3′-TTACTATGCCGCTGGTGGCTCTAGATGTGAGAAAGGGATGT
GCTGCGAGAAGGCTAGA[BC6](N)10(A)30BN-Frag-GACAGA
GAATATGTGTAGAGGCTCGGGTGCTCTG[i7]TAGAGCATACGGCAG
AAGACGAAC-5′

### Sequencing

### Read 1 [BC6]+UMI (N)10→

5′-
AATGATACGGCGACCACCGAGATCTACACTCTTTCCCTACACGACGCTC
TTCCGATCT[BC6](N)10(T)30VN-Frag-
CTGTCTCTTATACACATCTCCGAGCCCACGAGAC[i7]ATCTCGTATGC
CGTCTTCTGCTTG-3′
3′-
TTACTATGCCGCTGGTGGCTCTAGATGTGAGAAAGGGATGTGCTGCGA
GAAGGCTAGA[BC6](N)10(A)30BN-Frag-
GACAGAGAATATGTGTAGAGGCTCGGGTGCTCTG[i7]TAGAGCATAC
GGCAGAAGACGAAC-5′

Read 2 Nextera Index [i7]→
←Read 3: 3′ end cDNA fragment

To start, diluted ERCC RNA Spike-In Mix (1 μl of 1:107 for D1/D2 or 1 μl of 1:106 for D3; Life Technologies) was added to each well, and the template switching reverse transcription reaction described above was carried out using a MMLV Reverse Transcriptase (here, either SmartScribe Reverse Transcriptase (D1/D2; Clontech) or Maxima H Minus Reverse Transcriptase (D3; Thermo Scientific)) with the template-switching oligonucleotide (2 pmol, Eurogentec) (5′-iCiGiCACACTCTTTCCCTACACGACGCrGrGrG-3′ (SEQ ID NO: 17), where iC is iso-dC, iG is iso-dG, and rG is RNA G) and a cDNA synthesis primer (2 pmol, Integrated DNA Technologies) and 5′-/5Biosg/ACACTCTTTCCCTACACGACGCTCTTCCGATCT[BC6] NNNTTTTTTTTTTTTTTTTTTTTTTTTTTTTTTVN-3′ (SEQ ID NO: 18), wherein 5Biosg represents 5′ biotin; V represents a nucleotide selected from A, G, and C; the 3′ N represents a nucleotide selected from A, G, C, and T; [BC6] represents a 6 base pair barcode sequence; and the (N)10 after the barcode sequence represents a Unique Molecular Identifier (UMI) sequence (10 base pair barcode). After the template switching reaction, cDNA from 384 wells was pooled together and purified and concentrated using a single DNA Clean & Concentrator-5 column (Zymo Research). Pooled cDNAs were treated with an exonuclease, in this example Exonuclease I (New England Biolabs), and subsequently amplified by single primer PCR using the Advantage 2 Polymerase Mix (Clontech) and the SINGV6 primer (10 pmol, Integrated DNA Technologies) (5′-/5Biosg/ACACTCTTTCCCTACACGACGC-3′ (SEQ ID NO: 19)). Full length cDNAs were purified with Agencourt AMPure XP magnetic beads (0.6×, Beckman Coulter) and quantified on the Qubit 2.0 Flurometer using a dsDNA HS Assay (Life Technologies). The full-length cDNA was then used in the Nextera XT library preparation kit (Illumina) according to the manufacturer's protocol, with the exception that the i5 primer was replaced by a phosphorothioate bond-containing nucleic acid (5 μM, Integrated DNA Technologies) (5′-AATGATACGGCGACCACCGAGATCTACACTCTTTCCCTACACGACGCTC TTCCG*A*T*C*T*-3′, where *=phosphorothioate bonds (SEQ ID NO: 3)). The resulting sequencing library was purified with Agencourt AMPure XP magnetic beads (0.6×, Beckman Coulter), size selected (300-800 bp) on an E-Gel EX Gel, 2% (Life Technologies), purified using a QIAquick Gel Extraction Kit (Qiagen) and quantified on a Qubit 2.0 Flurometer using a dsDNA HS Assay (Life Technologies). Libraries were sequenced on an Illumina Hiseq paired-end flow cells with 17 cycles on the first read to decode the well barcode and UMI, an 8 cycle index read to decode the i7 Nextera barcode, and finally a 34 cycle second read to sequence the cDNA.

### Sequencing on Bulk Samples

Populations of both unsorted and sorted cells were lysed in QIAzol (Qiagen) and RNA was extracted and purified using Direct-zol RNA MiniPrep (Zymo Research). Digital gene expression (DGE) libraries for sequencing were prepared from 10 ng of extracted total RNA, using the protocol described above for single cells, with the exception of using more concentrated template-switching and barcoded nucleic acids (10 pmol) and a version of the cDNA synthesis primer that did not contain the well-specific 6 bp barcodes but instead a 16 bp UMI (Integrated DNA Technologies) (5′-/5Biosg/ACACTCTTTCCCTACACGACGCTCTTCCGATCT NNNN NNNNNTTTTTTTTTTTTTTTTTTTTTTTTTTTTTTVN-3′ (SEQ ID NO: 404))

### Single Cell RT-qPCR

Single cells were sorted into 384-well plates, frozen at −80° C., thawed for 5 min at room temperature, treated with proteinase K (200 μg/mL, Ambion), and desiccated as described above. cDNA synthesis was carried out in each well using SuperScript VILO (2 μl final volume; Life Technologies). qPCR was then performed on the total cDNA output using FAM and VIC Taqman probes (Life Technologies) and processed on an Applied Biosystems ViiA 7 Real-Time PCR system (Life Technologies).

### Single-Molecule FISH

Probes targeting LPL, G0S2 and TCF25 transcripts were synthesized as amine-conjugated oligonucleotides and then labelled with Cy5 (GE Healthcare), Alexa Fluor 594 (Molecular Probes) or 6-TAMRA (Molecular Probes). Hybridizations and washes were performed using modifications to previously described procedures (see, e.g., Bienko et al., Nat. Methods 10:122-124 (2013) and Raj et al., Nat. Methods 5:877-879 (2008)). Prior to hybridizations, lipids were extracted by incubation of fixed cells in 2:1 chloroform:methanol for 30 min at room temperature. Cells were washed quickly with 70% ethanol and then resuspended in 200 μl RNA Hybridization buffer containing 2×SSC buffer, 25% Formamide, 10% Dextran Sulphate (Sigma), E. coli tRNA (Sigma), Bovine Serum Albumin (Ambion), Ribonucleoside Vanadyl Complex and 150 ng of each desired probe set (the mass refers only to pooled oligonucleotides, excluding fluorophores, and is based on absorbance measurements at 260 nm). Hybridizations were performed for 16-18 h at 30° C., after which cells were washed twice for 30 min at 30° C. in RNA Wash buffer (containing 2×SSC buffer, Formamide 25% (Ambion) and 100 ng/ml DAPI). For microscopy, cells were resuspended in a mounting solution containing 1×PBS 0.4% Glucose, 100 μg/ml Catalase, 37 μg/ml Glucose Oxidase and 2 mM Trolox and immobilized on poly-lysine coated chambered cover glasses. Imaging was performed as described above, using an inverted epi-fluorescence microscope (Nikon) equipped with a high-resolution CCD camera (Pixis, Princeton Instruments) and a 100× magnification oil immersion, high numerical aperture Nikon objective. An image stack consisting of 50 image planes spaced 0.3 μm apart was acquired per region of interest. Individual images were filtered with a high-pass Fast Fourier Transform filter, where the filter cutoff was chosen to preserve diffraction-limited signals. Filtering was repeated on the resulting image of the maximum projection. Signal positions, widths, and intensities were quantified by fitting 2D Gaussians approximating the point-spread function (PSF) of the microscope. To separate sporadic signals caused by autofluorescence or non-specifically bound probes from real mRNA signals, signals were filtered based on width and signal-to-noise ratio. Cells were segmented manually and signals were assigned to individual cells.

### Computational Analysis of Sequence Data

All second sequence reads were aligned to a reference database containing all human RefSeq mRNA sequences (obtained from the UCSC Genome Browser hg19 reference set), the human hg19 mitochondrial reference sequences and the ERCC RNA spike-in reference sequences, using bwa version 0.7.4 4 with non-default parameter “−1 24”. Read pairs for which the second read aligned to a human RefSeq gene were kept for further analysis if 1) the initial six bases of the first read all had quality scores of at least 10 and corresponded exactly to a designed well-barcode and 2) the next ten bases of the first read (the UMI) all had quality scores of at least 30. Digital gene expression (DGE) profiles were then generated by counting, for each microplate well and RefSeq gene, the number of unique UMIs associated with that gene in that well. Python scripts were used to implement the alignment and DGE derivation from the samples.

### Computational Analysis of DGE Profiles

All computational and statistical analyses were performed using Python 2.7 with the Enthought Canopy Distribution, Numpy 1.8.0 and Scipy 0.13.0, scikit-learn 0.14, and Matplotlib 1.3.1. For each plate, wells with less than 1,000 or more than 10,000 total UMI counts were discarded (24% of all wells, largely low-value wells). The UMI counts for each gene in the remaining wells were then normalized by dividing by the sum of UMI counts across all genes in the same well. This normalization removes variation from differences in RNA content per cell and can be revisited for analyses that are sensitive to this phenomenon. Pairwise Pearson correlations between genes across single cells and their associated p-values were computed using the scikit-learn metrics.pairwise_distances function. The 5% false discovery rate (FDR) thresholds were estimated from the p-value distribution using the Benjamini-Hochberg-Yukeli procedure. The expected null distributions of pairwise correlation coefficients were estimated by permuting expression values across cells from the same time point and re-computing the pairwise correlations 100 times. Principal component analyses (PCA) were performed by first scaling the normalized UMI-derived expression levels of each gene to zero mean and unit variance using the scikit-learn preprocess.scale function and then applying the RandomizedPCA transformation. Each time course dataset was processed separately. To project lipid-sorted cell data into the corresponding time course principal component space (i.e., the three dimensional space represented by the 3 major principal components), the time course and lipid-sorted expression values were concatenated and re-scaled prior to applying the time course PCA transformation. Gene set enrichment analyses (GSEA) were performed using the GSEAPreRanked module of the GSEA 2.0 software (http://www.broadinstitute.org/gsea/) with the MSigDB 4.0 gene sets 6. Genes were ranked by the PC weights for interpretation of PC metagenes or by the signal to noise metric (μA-μB/σA-σB) for comparisons of low and high lipid cells. Significant gene sets were called at the threshold recommended by the GSEA developers (25% FDR).

### Results

A variety of cell populations can be induced to differentiate into adipocytes by treating the cells with cocktails of adipogenic hormones and growth factors. However, the yields of lipid-filled, adipocyte-like cells obtained from these methods are highly variable. Moreover, it is unclear whether this variability reflects heterogeneity in the starting populations, stochastic responses to imperfect differentiation stimuli, or other factors. Thus, adipocyte differentiation was selected as a good model system to test single-cell sequencing. The most commonly used cell line in adipogenesis research is the immortalized murine 3T3-L1 cell line, which supports near complete conversion to adipocyte-like cells. Numerous molecular differences have, however, been found between this cell line and human adipocyte stem cells (hASCs). Single-cell profiling should help clarify the nature of these differences.

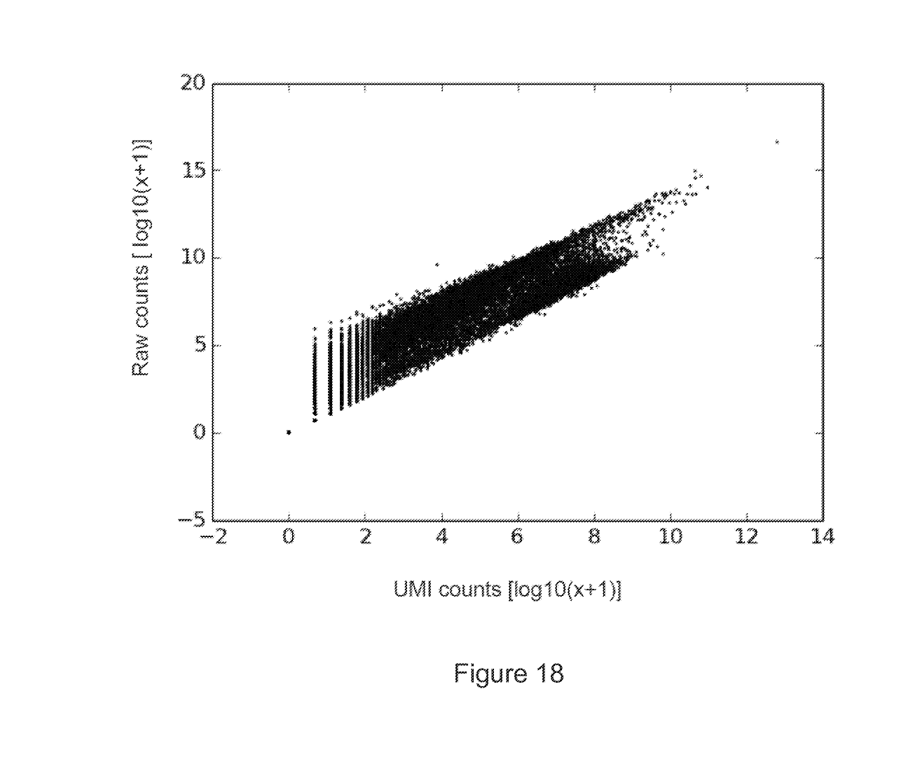

hASC cultures were collected just prior to induction of differentiation (day 0), as well as at seven time points after induction (days 1, 2, 3, 5, 7, 9 and 14). At day 14, approximately two thirds of the cells contained clearly visible lipid droplets while the remainder retained a more fibroblastlike morphology. A nucleic acid stain was used to identify and sort intact single cells into 384-well plates with a fluorescence-activated cell sorter. A neutral lipid stain also was used to separately sort single cells based on their lipid contents. This method allowed us to combine the advantages of FACS sorting, such as staining cells using, for example, a DNA stain or a lipid stain, and selecting specific cells to profile. Additional cells then were collected and sorted from independent cultures at days 0, 3 and 7. In total, single-cell sequencing libraries were prepared from 44 microplates. The plates were sequenced to a mean depth of ˜165,000 reads per well and the reads aligned to RefSeq transcripts. After stringent filtering on sequence and alignment quality, and then estimating the expression levels in each cell from UMI counts (FIG. 18), survey-depth digital gene expression (DGE) profiles were obtained from a total of 12,832 cells (76% of the total wells). As judged by the UMI counts, each DGE profile captured between 1,000 and ˜10,000 unique mRNAs (mean=2,602 and 3,336 for the protocols from Example 1 and this Example, respectively), which constitutes a ˜4-fold increase in mean library complexity relative to a previous high-throughput protocol (Jaitin et al., Science 343:776-779 (2014)).

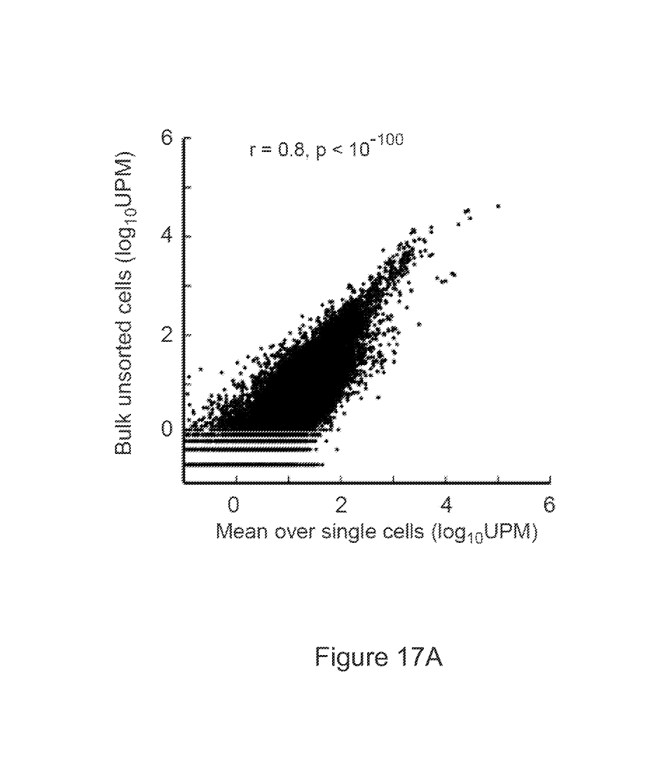

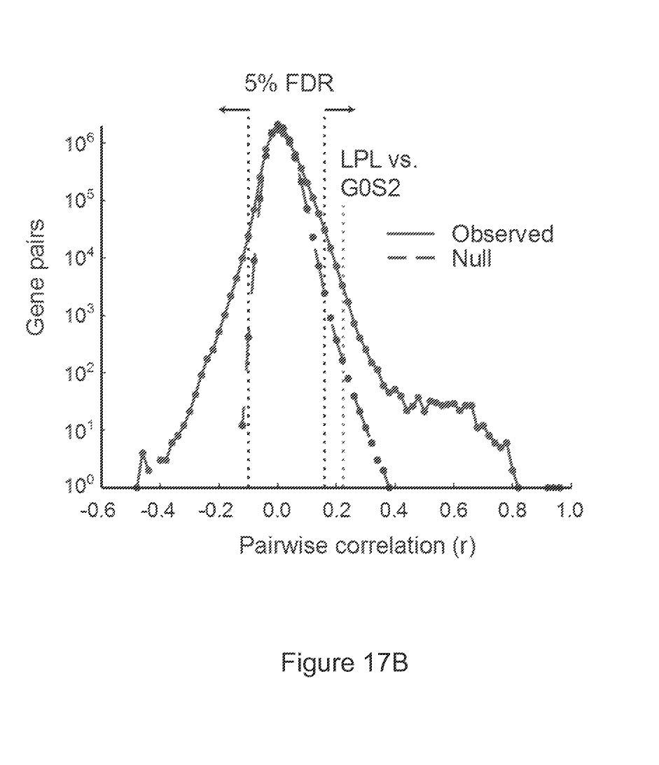

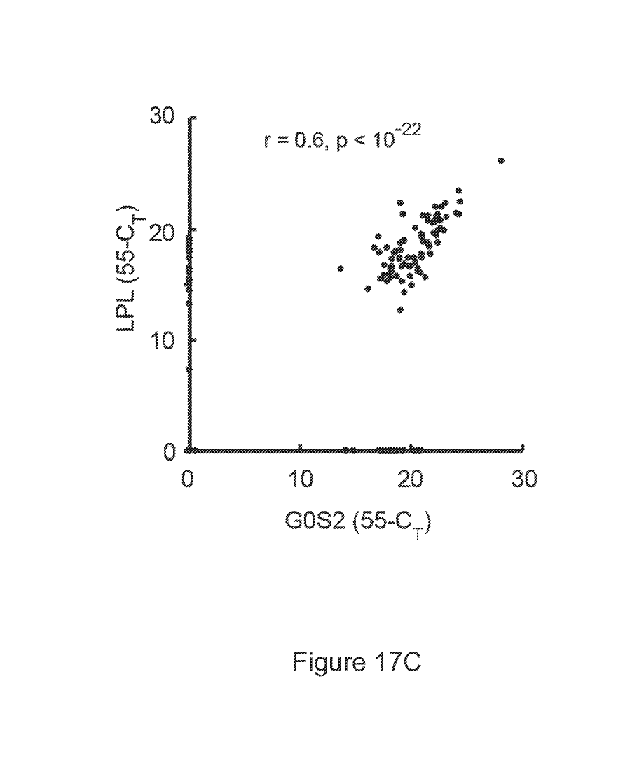

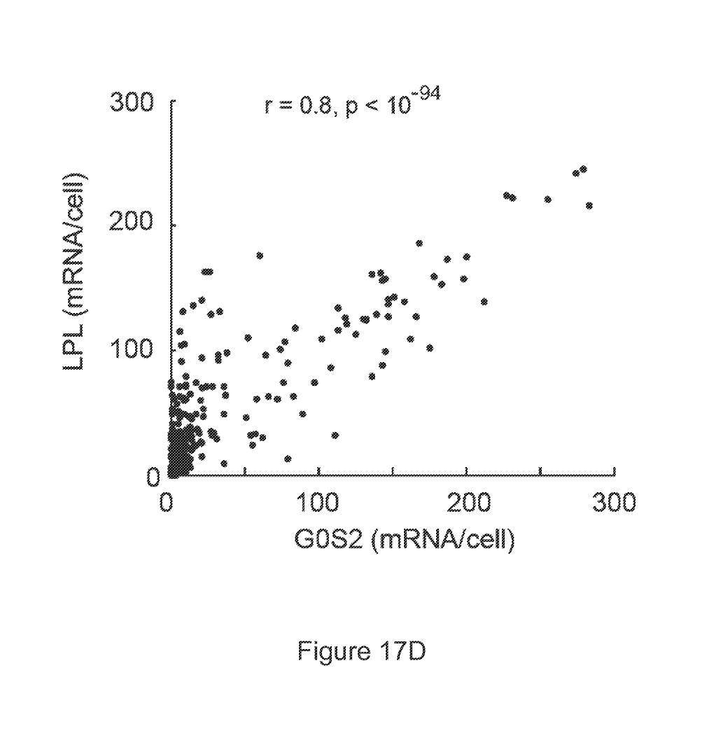

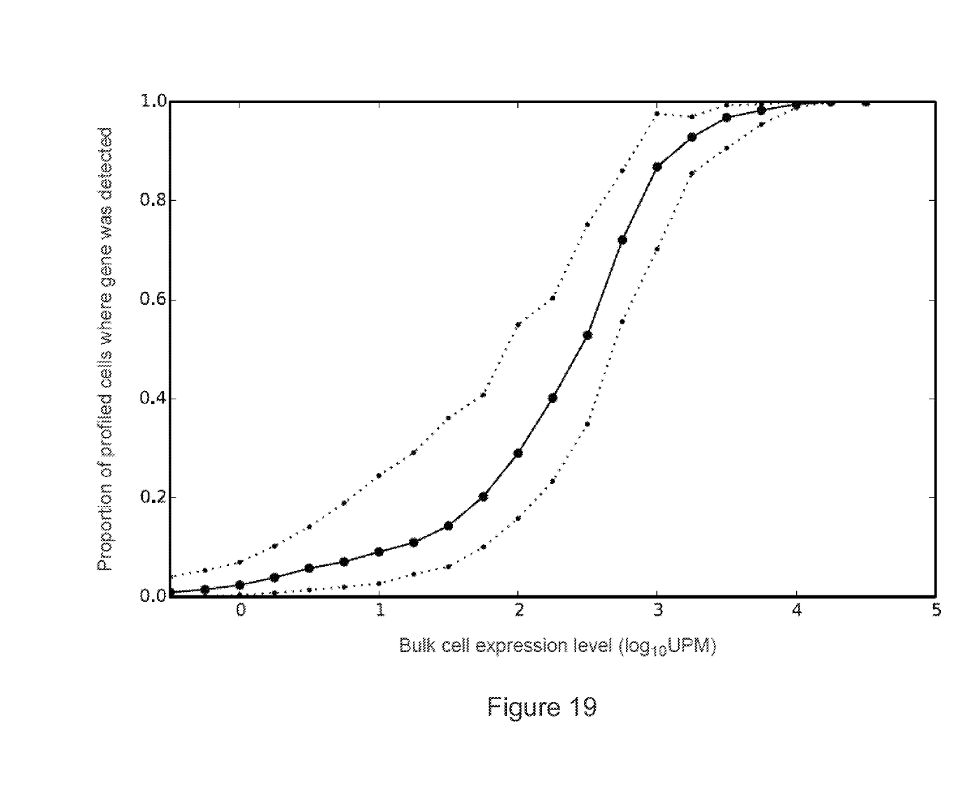

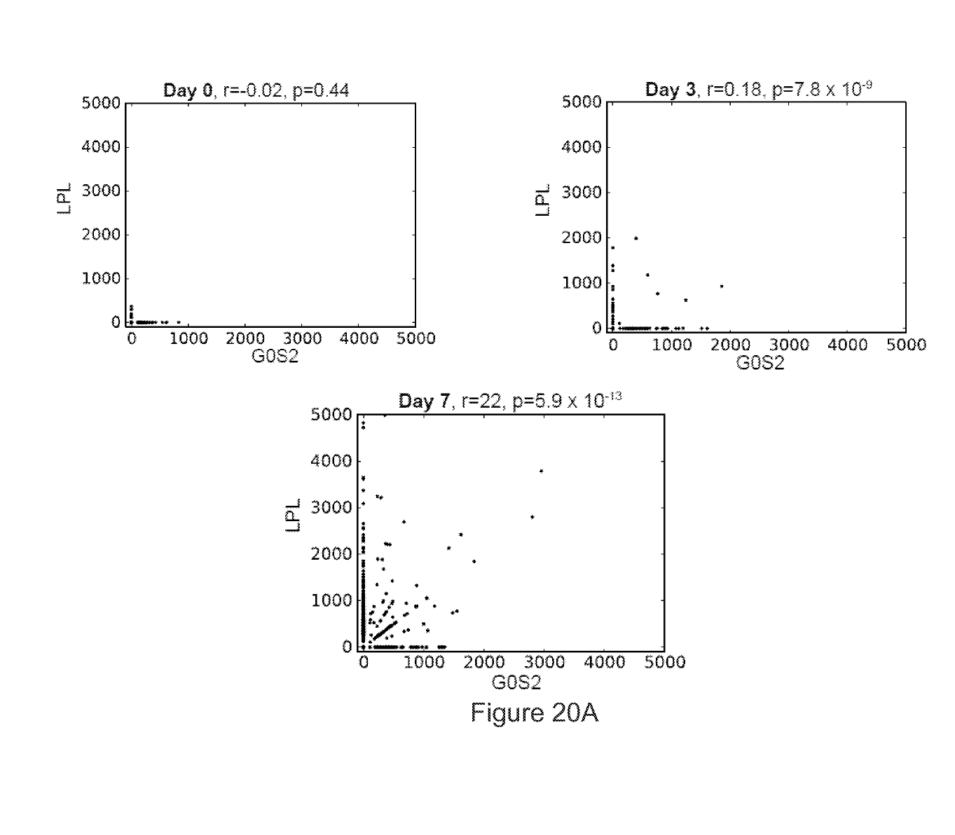

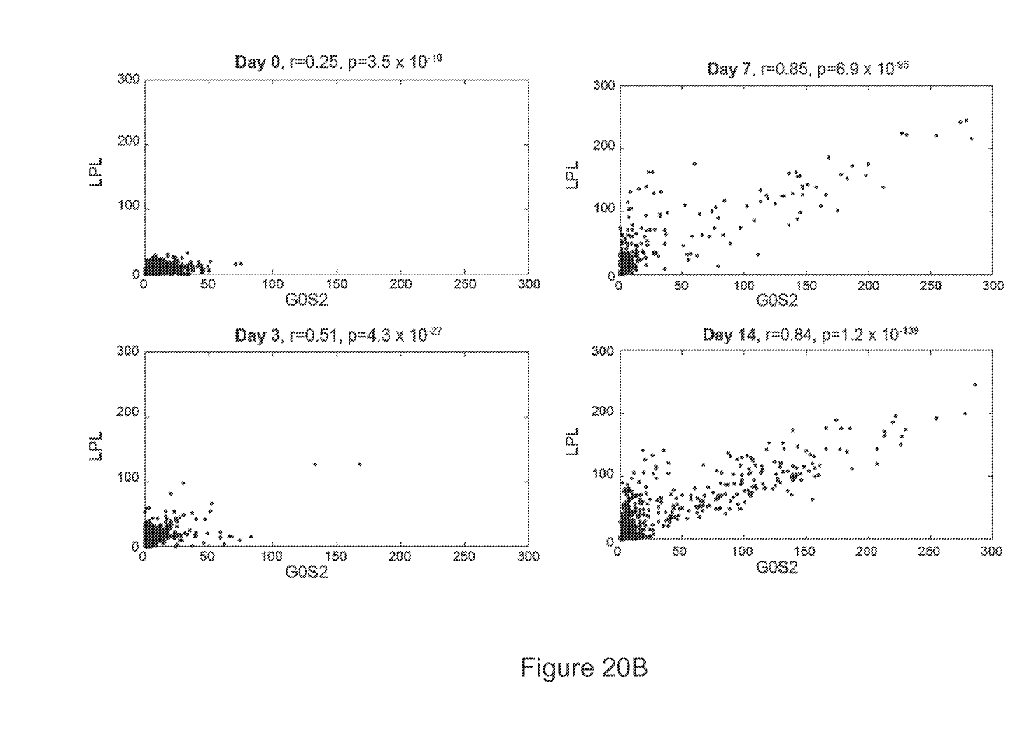

Initial analysis of the resulting data showed that the mean gene expression levels across the single cell profiles were significantly correlated with their corresponding levels from bulk unsorted cells collected at the same time point (r=0.8, p<10-100; FIG. 17A). Of 15,099 distinct RefSeq genes that were detected at day 0 in bulk unsorted cells, 14,612 (97%) also were detected in at least one single cell from the same day. As expected from the relatively low sequencing coverage, only the most actively transcribed genes were captured from every cell (FIG. 19). However, significant positive and negative correlations still could be detected between the expression levels of individual genes across cells collected on the same day (FIG. 17B). For example, LPL and G0S2, two traditional markers that are both up-regulated after induction of adipogenesis, had positively correlated expression levels after differentiation (r=0.23, p<10-12 on day 7; FDR≦5%). A positive correlation could be validated between these genes both by qRT-PCR analysis of independently sorted single cells (FIG. 17C) and in situ by multiplexed single molecule FISH (smFISH; FIG. 17D and FIG. 20). Thus, the single cell RNA sequencing method tested can capture gene expression variation at single-cell resolution.

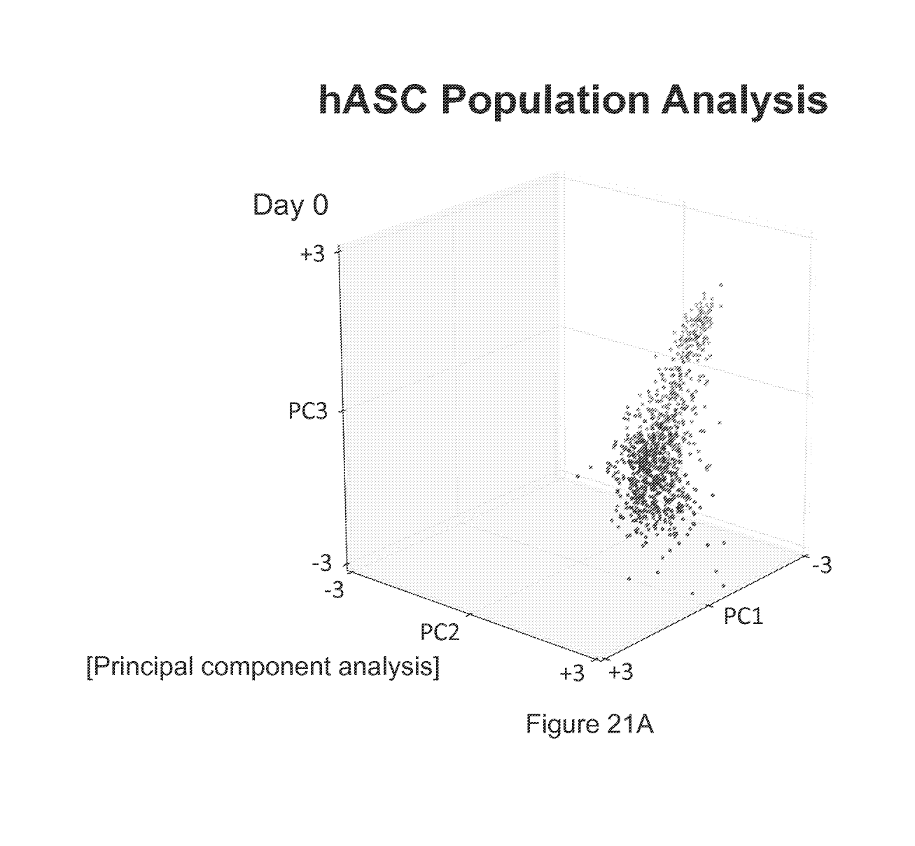

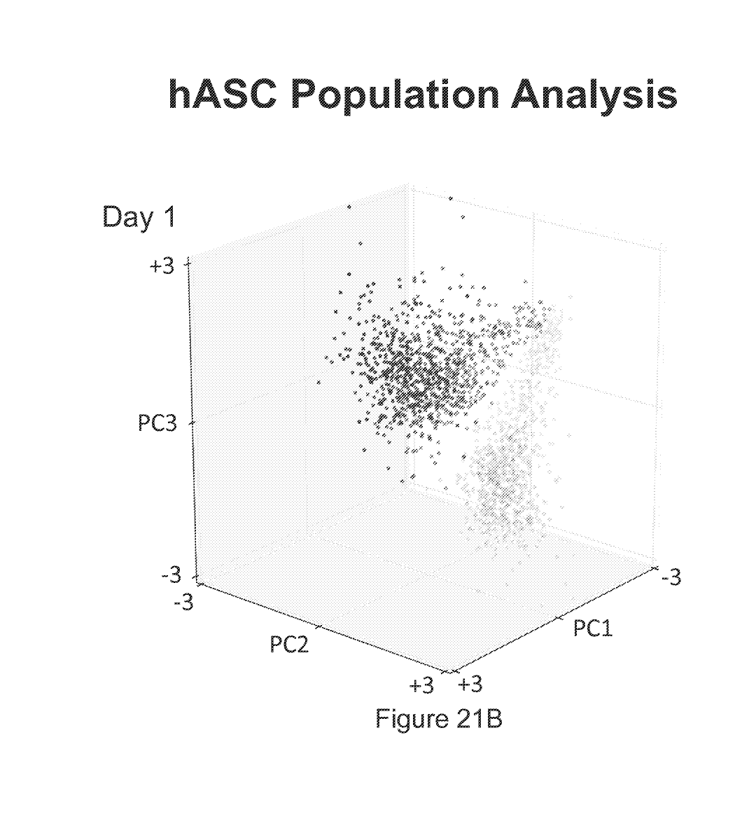

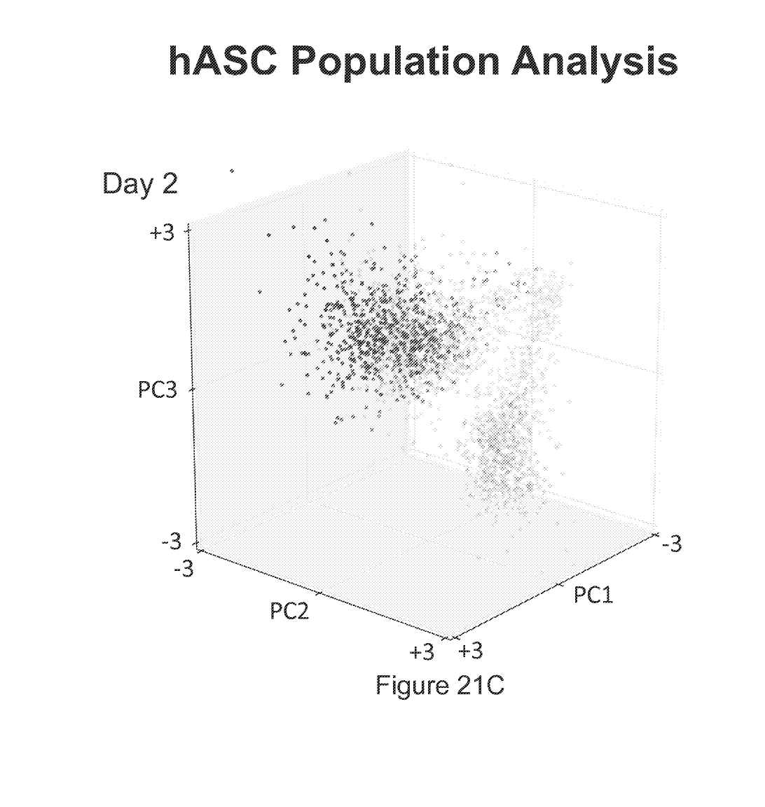

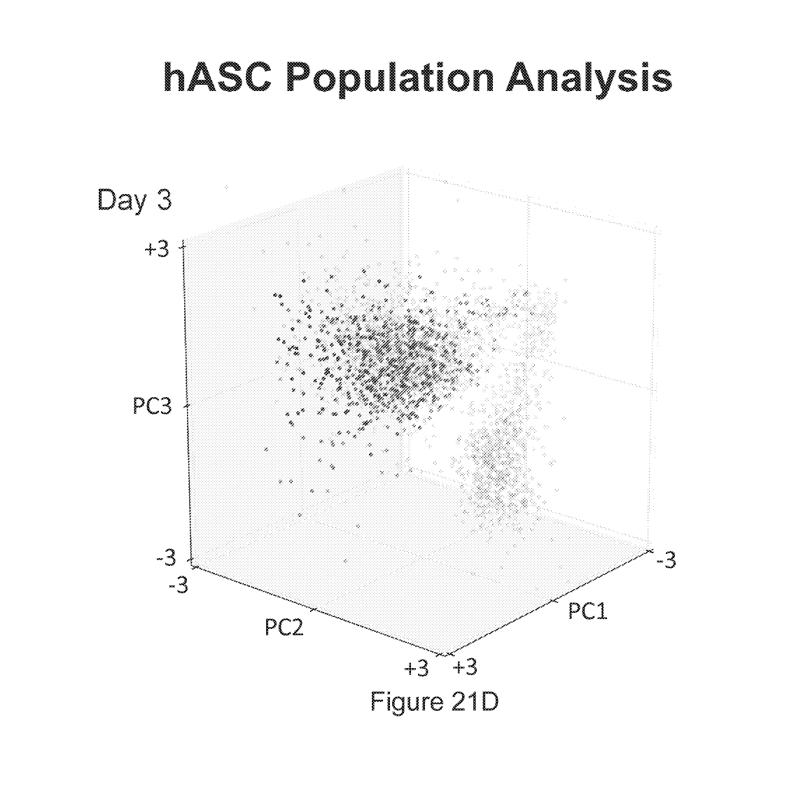

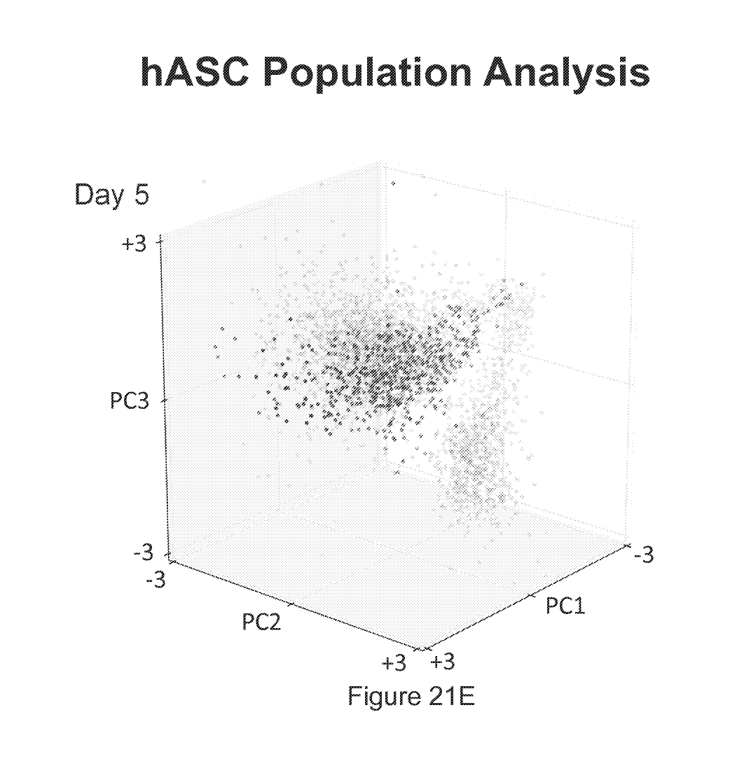

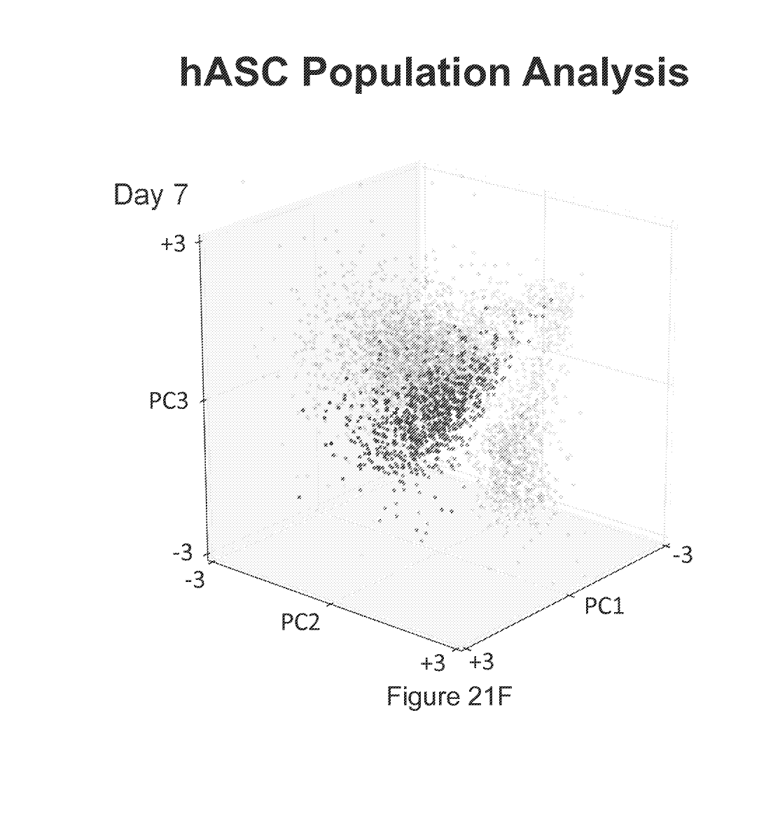

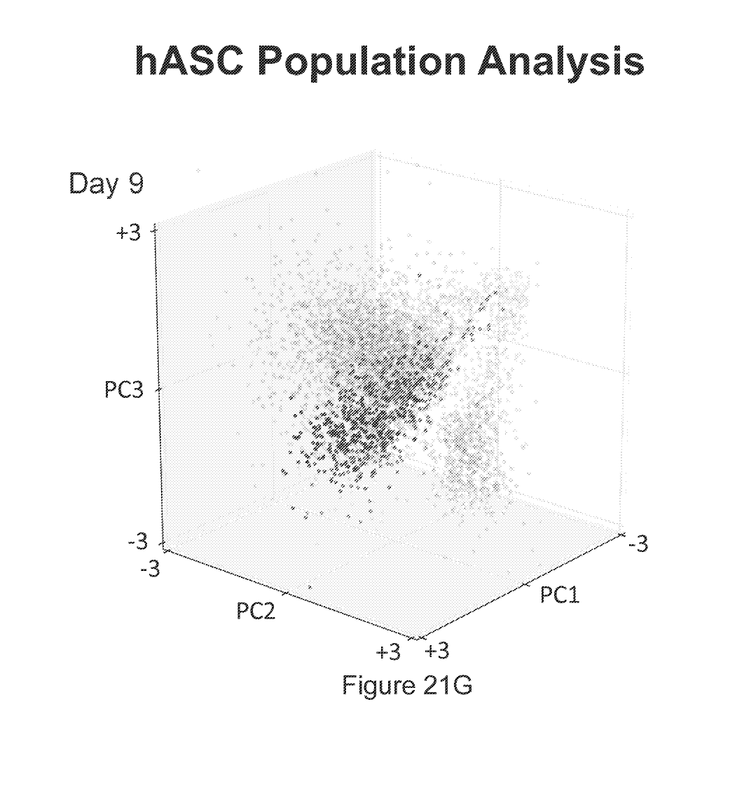

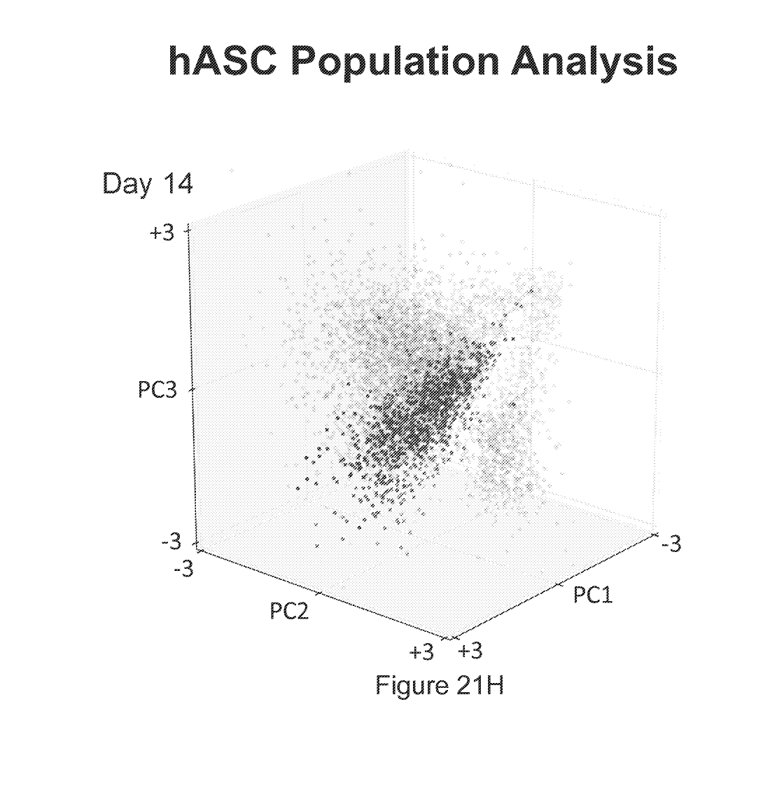

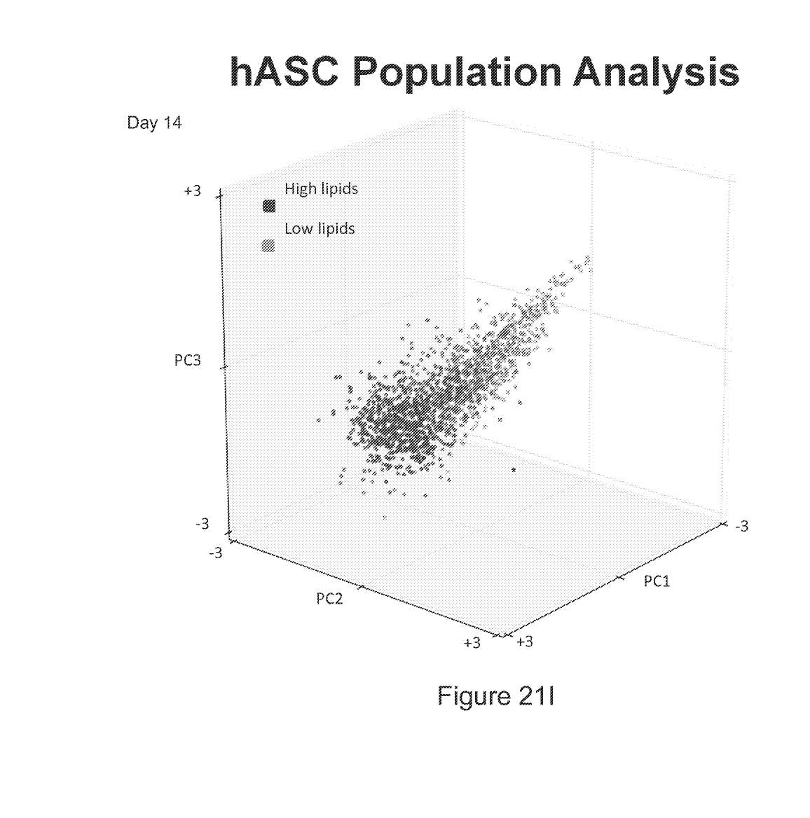

To understand the observed cell-to-cell variation in gene expression in more detail, a principal component analysis (PCA) of the initial time course (days 0 to 14; 6,197 cells; FIG. 21A-H) was performed. Plotting the position of each cell in the space defined by the first three principal components revealed that there was little overlap between cells from day 0 and cells from later time points. This suggested that addition of the adipogenic differentiation cocktail induced a rapid response in virtually all of the cultured cells. Plotting the positions also revealed that gene expression levels continued to evolve from day 1 to day 14, but that there was substantial overlap between the cells collected at close time points. This is consistent with a population-wide, but asynchronous, response to induction of differentiation.

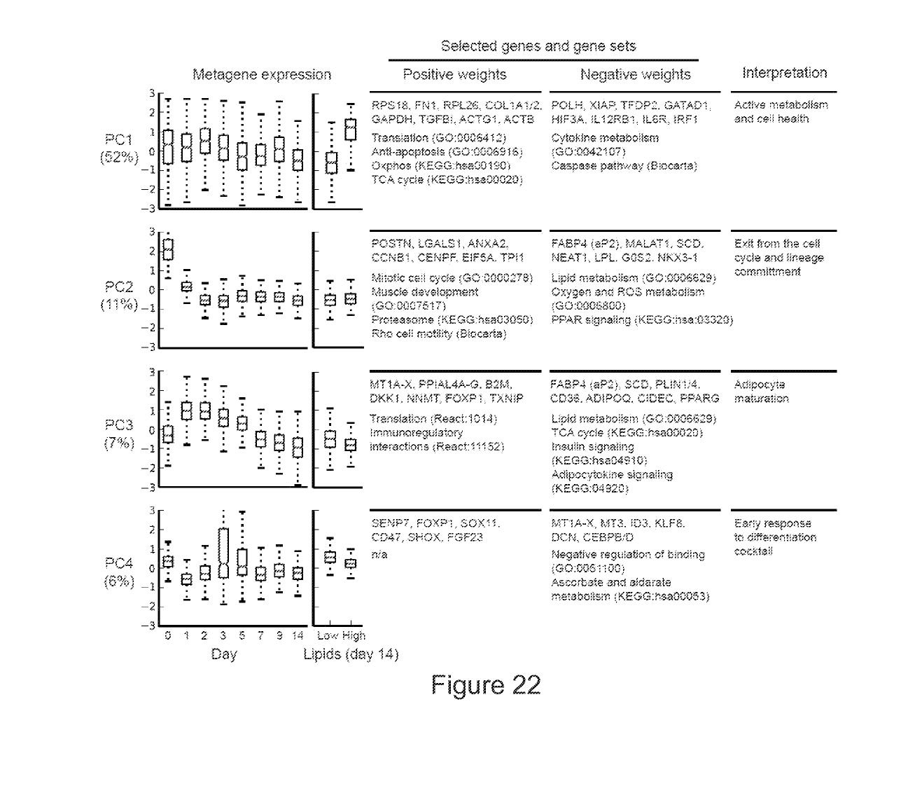

To explore the biological basis for the observed gene expression variation, the relationships between each of the top principal components (PCs), gene expression and time, were then examined (FIG. 22). The PCs can be interpreted as metagenes that capture coordinated expression of multiple genes in the original data set. For each PC, we therefore ranked the genes according to their corresponding PC weights and then looked for evidence of coordinately regulated pathways using gene set enrichment analysis (GSEA). This analysis suggested qualitative biological interpretations for at least the top four PCs.

The first PC metagene (PC1) was positively associated with genes involved in general cellular metabolism, including the majority of genes involved in ribosome assembly, mitochondrial biogenesis, and oxidative phosphorylation, while it was negatively associated with inflammatory pathways, cytokine production and caspase expression. Variations along PC1 reflect differences between metabolically active “healthy” and inactive “unhealthy” cells. Interestingly, while there was a shift towards the latter state towards day 14, there was substantial overlap between the PC1 distributions from all time points, which indicates that this axis of variation was a major contributor to culture heterogeneity prior to induction of differentiation. Because significant cell detachment or death was not observed during the two weeks of differentiation, the inflammation signature likely represents a chronic cell state rather than ongoing apoptosis. By contrast, PC2 was high only in cells collected from day 0, effectively separating these from the differentiating cells. It showed a strong positive association with expression of genes required for progression through the mitotic cell cycle and, to a lesser extent, with genes associated with non-adipogenic differentiation. A decrease in PC2 may therefore reflect an exit from the cell cycle and lineage commitment. Expression of PC3 was high during the first two days post-induction, but steadily decreased as the cells approached day 14. This decrease was associated with up-regulation of lipid homeostasis pathways and markers of adipocyte maturation. PC4 showed a transient drop at day 1, which was associated with increased expression of genes known to be rapidly induced by adipogenic cocktails, including early adipogenic regulators CEBPB and CEBPD 11. PC4 may therefore reflect an early response to induction of differentiation.

To explore the relationship between variations in gene expression and in lipid droplet accumulation, an additional 933 cells with high lipid content and an additional 666 cells with low lipid content were collected and analyzed at day 14. When the DGE profiles of these cells were projected into the space defined by the initial time course PCs, the high and low lipid cells were largely separated by their distribution along PC1 (FIG. 21I and FIG. 22). Particularly, cells with higher lipid content showed higher expression of genes related to basic cellular metabolism, while cells with lower lipid content showed higher expression of inflammatory genes. Interestingly, there was substantial overlap along PC3, and while some classic adipocyte markers like FABP4 (aP2) were enriched in the high lipid fraction, key regulatory factors such as PPARG were not. This implies that pathways related to lipid homeostasis and adipocyte maturation had been activated in both fractions.

Separate PCAs of the second collected time course (2,968 cells from days 0, 3 and 7, and 2,068 additional cells with high or low lipids from day 7) yielded qualitatively similar patterns, which suggests that the observations are robust to technical variation across cell cultures. Thus, while morphological analysis suggested that only a fraction of hASCs respond to the differentiation cocktail, the single-cell data surprisingly show that virtually all of the cells exited the mitotic cell cycle and proceeded to up-regulate an adipogenic gene expression program. The observed variability in lipid droplet accumulation and conversion to mature adipocyte-like morphologies is instead most strongly linked to an inverse correlation in expression of basic cellular metabolism and inflammatory expression programs, which was also present prior to the induction of differentiation. Notably, cells with low lipid contents showed elevated expression of several pro-inflammatory regulatory factors, including IRF1, IRF3 and IRF4. These factors have previously been shown to negatively influence total lipid accumulation in murine bulk cultures and in vivo models, which supports a causal link between cell-to-cell variation in expression of these factors and lipid accumulation. Specific activation in the fraction of low lipid cells may explain the paradoxical increases in expression of these factors that have previously been observed in bulk cultures.

### Example 4

### Protocol for High Throughput Sequencing

Although the protocols described above were originally designed to perform RNA sequencing on sorted single cells, they are also suitable for use with other starting samples, such as extracted or purified RNA (bulk RNA sequencing) or a population cells or tissues (e.g., cell or tissue lysates). As with single cell RNA sequencing, using a 3′ digital gene expression method allows the profiling of a high number of samples in a cost-efficient manner. The protocol is robust for a broad range of input from single cells to pooled cells or extracted RNA. It allows the profiling of a large number of samples of extracted RNA (patient samples for example), profiling of a population of small number of cells (e.g., cell or tissue lysates), as well as analysis of sorted, single cells. Regardless of starting materials, the use of the barcodes and UMIs described herein permit the tracking of individual transcripts to a specific multi-well plate and to a specific well of that plate, thus permitting correlation of data to the original starting material. The above examples are indicative of the powerful applications of the technology.

By way of further example, the ability to correlate expression analysis to a particular well of a multi-well plate (e.g., to the starting sample) is critical in the screening assay context, regardless of whether the material in the screen is a single cell or lysate. Because the bar codes and UMI allow tracking of individual transcripts, sequencing reactions can be run as massive multiplex reactions rather than a series of individual reactions without losing transcript-level data. This results in a significant increase in efficiency and decrease in cost. The sequencing data then can be deconvoluted using, for example, 3′ digital gene expression to count the number of occurrences of bar code and UMI sequences and obtain an expression level for a particular transcript.

The methods and reagents described herein also are adaptable to other platforms, e.g., microfluidic systems such as Fluidigm's Cl microfluidic device. For example, the capture of 96 cells was performed on the Cl chip, and the reagents and adapters to prepare the cDNA were incorporated directly on the Cl chip. cDNAs were retrieved as an output of the Cl chip, pooled, and prepared as a Nextera library.

The nucleic acids, methods, and kits of the invention also provide the ability to profile single cells for which it is not possible to do an individual RNA extraction and purification, or, by working directly with lysates, profiling a high number of conditions under which cells are cultivated without necessarily performing a separate RNA extraction and purification step (e.g., if sequencing cells from a high throughput compound screen, it is unnecessary to extract and purify the RNA from each well individually).

In certain embodiments, one or more of the following modifications to the protocol or reagents used were and can optionally be employed. Specifically, another reverse transcriptase can be used, such as the MMLV Maxima H Minus Reverse Transcriptase (Thermo Scientific). At this point, numerous different MMLV reverse transcriptases have been successfully used and can be selected based on user preference, cost, availability and the like. In certain embodiments, a proteinase or protease, such as proteinase K, may be added during lysis. In certain embodiments, proteinase K is included as part of lysis for sorted single cells and isolated cells/lysates. Higher concentrations of proteinase K and increased incubation times are used, in certain embodiments, for a pool of cells as compared to single cells. Other modifications include a reduction in the volume of the RT reaction to 2 μl by drying out the RNA during the proteinase K inactivation to increase reaction efficiency and use of 6-nucleotide barcodes to refer to a sample or pool instead of a single cell when performing sequencing on extracted RNA or a pool of cells.

For bulk RNA sequencing, 10 ng of total RNA were used as input, although this amount is flexible. Additionally, reactions were performed in 10 μl, and the reactions used more concentrated (10 μM) template-switching and barcode-containing oligonucleotides. For RNA sequencing of lysates, inputs ranged from single cells to 10,000 cells (including tens or hundreds of cells). For pooled cells, more concentrated proteinase K (2 mg/ml instead of 1 mg/ml for single cells) was used, and the cells were incubated longer (one hour at 50° C. instead of 15 minutes) to increase lysis efficiency.

An exemplary protocol is as follows.

### Capture Plate Preparation

Add 54 of lysis buffer, composed of a 1/500 dilution of Phusion HF buffer (New England Biolabs, #B0518S) in each well of a collection Twin.tec PCR 384-well plate (Eppendorf, #951020729).

### Cell Preparation

Remove media by pelleting the cells (5 min at 1000 rpm), and resuspend the cells in RNAprotect Cell Reagent (˜1004 per 100,000 cells, Qiagen, #76526) and 1 μL of RNaseOUT Recombinant Ribonuclease Inhibitor (Life Technologies, #10777-019). Cells can be stored up to 2 weeks at 4° C. Next, dilute the cells in ˜1.5 mL PBS, pH 7.4 (no calcium, no magnesium, no phenol red, Life Technologies, #10010-049). Stain the cells for viability (DNA staining by Hoechst 33342) with NucBlue Live ReadyProbes Reagent (Life Technologies, #R37605).

### Cell Collection

Sort individual cells in each well of the 384-well capture plate using the FACSAria II flow cytometer (BD Biosciences). “Live” cells are selected and duplets avoided using the Hoechst DNA staining After sorting, immediately seal the plates, spin them down, and freeze them on dry ice. Sorted cells are stored at −80° C. If performing bulk lysate sequencing, which starts with extracted/purified RNA and proceeds directly to reverse transcription/template switching, this step should be skipped.

### Cell Lysis

Thaw the cells for 5 minutes at room temperature, then place the plate on ice. Add 1 μL of Proteinase K Solution (diluted to 1 mg/mL; 1/20; LifeTechnologies, #AM2548) to each well. Incubate the plate at 50° C. for 15 minutes, then remove the seal and incubate the plate at 95° C. for 10 minutes. Place the plate back on ice.

### Reverse Transcription/Template Switching

Denature 42 μl of a 1×10⁻⁶ dilution of ERCC RNA Spike-In Mix (Life Technologies, #4456740) for 2 min at 70° C., then place directly on ice. Prepare the following RT/template switching mix (for 384 wells): 160 μl of 5×RT buffer, 80 μl of dNTPs (New England Biolabs, #N0447L), 72 μl of Nuclease-Free Water (not DEPC-Treated) water (LifeTechnologies, #AM9937), 40 μl of a denatured 1×10⁻⁶ dilution of ERCC RNA Spike-In Mix (Life Technologies, #4456740), 8 μl of the universal E5V6NEXT adapter (100 μM, Eurogentec), and 50 μL of Maxima H Minus Reverse Transcriptase (Thermo Scientific, #EP0753). Add 1 μl of the mix to each well and 1 μL of the barcoded oligonucleotide adapter (2 μM, Integrated DNA Technologies to each well. Incubate the plate at 42° C. for 1 hour 30 minutes.

cDNA Pooling and Purification

Pool all 384 wells together, and add 5.5 mL of DNA Binding Buffer (Zymo Research, #D4004-1-L) to the pooled cDNAs. Purify all cDNAs pooled from one 384-well plate through one single DNA Clean & Concentrator-5 column (Zymo Research, #D4013). Elute cDNAs in 18 μL of Nuclease-Free Water.

### Exonuclease I Treatment

Add 2 μL of 10× reaction buffer and 1 μL of Exonuclease I (New England Biolabs, #M0293L) to the cDNAs. Incubate the reaction at 37° C. for 30 minutes, then at 80° C. for 20 minutes.

Full Length cDNA Amplification

Amplify full length cDNA by single primer PCR using the Advantage 2 PCR Enzyme System (Clontech, #639206). The PCR reaction is as follows: 200 μL of cDNA from previous step, 54 of 10× Advantage 2 PCR buffer, 1 μL of dNTPs, 1 μL of the SINGV6 primer (10 μM, Integrated DNA Technologies), 1 μL of Advantage 2 Polymerase Mix, and 224 of Nuclease-Free Water. Perform the PCT according to the following program: 95° C. for 1 minutes; 18 cycles of a) 95° C. for 15 seconds, b) 65° C. for 30 seconds, and c) 68° C. for 6 minutes; 72° C. for 10 minutes; and, optionally, 4° C. to store the reaction.

Full Length cDNA Purification and Quantification

Purify the full length cDNAs with 304 of Agencourt AMPure XP magnetic beads (Beckman Coulter, #A63880). Elute the full length cDNAs in 124 of Nuclease-Free Water and quantify on the Qubit 2.0 Flurometer (Life Technologies) using the dsDNA HS Assay (Life Technologies. #Q32851).

### Sequencing Library Preparation

To increase complexity, all cDNA from the purified full length cDNA is engaged in the Nextera library preparation. If the total amount of cDNA is superior to 1 ng and inferior to 10 ng, proceed to tagmentation reactions of ˜1 ng according to the Illumina Nextera XT (FC-131-1024) protocol. After the neutralization step, add 180 μl DNA Binding Buffer (Zymo Research, #D4004-1-L) to each tagmentation reaction, and pool and purify the tagmentation reactions on one single DNA Clean & Concentrator-5 column (Zymo Research, #D4013). Then, amplify the tagmented purified cDNA following the Illumina protocol with the exception of running only 10 cycles of PCR, using only the i7 primer to barcode cDNA originating from the same 384-well plate and replacing the i5 primer with PSNEXTPTS, 5 μM (Integrated DNA Technologies) as the second primer. If the total amount of cDNA is superior to 10 ng and inferior to 50 ng, proceed to the tagmentation using the Nextera DNA kit (FC-121-1030), suitable for SOng of input. Scale down all reagents and reaction volume according to the input concentration. Purify the tagmented cDNA on a single DNA Clean & Concentrator-5 column (Zymo Research, #D4013) according to the Illumina protocol. Use the 25 μl eluted cDNA for the library amplification, and use only the i7 primer to barcode cDNA originating from the same 384-well plate, replacing the i5 primer with P5NEXTPT5, 5 μM (Integrated DNA Technologies) as the second primer. Do not add the PCR primer cocktail. Perform either 10 cycles (for an input of less than 20 ng) or 5 cycles (for an input of 20 ng and above) of PCR according to the Illumina protocol.

### Sequencing Library Purification and Size Selection

Purify the sequencing library with 304 of Agencourt AMPure XP magnetic beads and elute it in 204 of water. Run the entire library on an E-Gel EX Gel, 2% (Life Technologies, #G4010-02) and excise, purify using the QIAquick Gel Extraction Kit (Qiagen, #28704), and elute in 15 μl the band corresponding to a size range of 300 to 800 bp.

### Sequencing Library Quality Assessment

Quantify the library on the Qubit 2.0 Flurometer using the dsDNA HS Assay. Optionally, the quality and average size of the library can be assessed by BioAnalyzer (Agilent) with the High Sensitivity DNA kit (Agilent, #5067-4626).

### Sequencing

Sequencing can be performed on any Illumina HiSeq or MiSeq, using the standard Illumina sequencing kit. Libraries are run on paired-end flow cells by running 17 cycles on the first end, then 8 cycles to decode the Nextera barcode and finally 46 cycles. Up to twelve Nextera libraries/384-well capture plate, each comprising 384 cells, can be multiplexed together (twelve i7 barcodes currently available) allowing the simultaneous sequencing of up to 4,608 single cell transcriptomes on a single lane.

Exemplary sequences are provided below and herein. Such sequences are merely illustrative of various polynucleotides and components useful in the methods of the present invention. These polynucleotides are suitable across any of the various sample types described herein (e.g., single cells, lysates, bulk RNA, etc.).

### Adapter/Primer Sequences

### Template-Switching Oligonucleotide

(SEQ ID NO: 17)
5′-iCiGiCACACTCTTTCCCTACACGACGCrGrGrG-3′

iC: iso-dC
iG: iso-dG

### rG: RNA G

### Bar Code-Containing Oligonucleotide Adapter

(SEQ ID NO: 18)
5′-/5Biosg/ACACTCTTTCCCTACACGACGCTCTTCCGATCT[BC6]
NNNTTTTTTTTTTTTTTTTTTTTTTTTTTTTTTVN-3′

5Biosg: 5′ biotin

### V: (A, G, or C)

### N: (A, G, C, or T)

[BC6]: 6 bp barcode, different in each well. The barcodes were designed such that each barcode differs from the others by at least two nucleotides, so that a single sequencing error cannot lead to the misidentification of the barcode. (N)10: Unique Molecular Identifier (UMI).

### Amplification Primer

(SEQ ID NO: 19)
5′-/5Biosg/ACACTCTTTCCCTACACGACGC-3′

5Biosg: 5′ biotin

### Phosphorothioate Bond-Containing Nucleic Acid

(SEQ ID NO: 3)
5′-AATGATACGGCGACCACCGAGATCTACACTCTTTCCCTACACGAC
GCTCTTCCG*A*T*C*T*-3′

*: phosphorothioate bond

1. A nucleic acid comprising a 5′ poly-isonucleotide sequence, an internal adapter sequence, and a 3′ guanosine tract.

2-6. (canceled)

7. The nucleic acid of claim 1, wherein the adapter sequence is 12 to 32 nucleotides in length.

8. The nucleic acid of claim 7, wherein the adapter sequence is 22 nucleotides in length.

9. The nucleic acid of claim 8, wherein the internal adapter sequence is 5′-ACACTCTTTCCCTACACGACGC-3′.

10. A nucleic acid comprising a 5′ blocking group, an internal adapter sequence, a barcode sequence, a unique molecular identifier (UMI) sequence, a complementarity sequence, and a 3′ dinucleotide sequence comprising a first nucleotide and a second nucleotide, wherein the first nucleotide of the dinucleotide sequence is a nucleotide selected from adenine, guanine, and cytosine, and the second nucleotide of the dinucleotide sequence is a nucleotide selected from adenine, guanine, cytosine, and thymine.

11. (canceled)

12. The nucleic acid of claim 10, wherein the 5′ blocking group is biotin.

13-14. (canceled)

15. The nucleic acid sequence of claim 12, wherein the internal adapter sequence is 5′-ACACTCTTTCCCTACACGACGC-3′.

16-22. (canceled)

23. A kit comprising the nucleic acid of claim 7.

24. The kit of claim 23, further comprising the nucleic acid of claim 10.

25-29. (canceled)

30. The kit of claim 23, further comprising a third nucleic acid primer comprising 12 to 32 nucleotides and a 5′ blocking group.

31-35. (canceled)

36. The kit of claim 23, further comprising a phosphorothioate bond-containing nucleic acid comprising an X1*X2*X3*X4*X5*3′ sequence, wherein * is a phosphorothioate bond.

37-38. (canceled)

39. The kit of claim 36, wherein the sequence of the phosphorothioate bond-containing nucleic acid is 5′-AATGATACGGCGACCACCGAGATCTACACTCTTTCCCTACACGACGCTCTTCCG*A*T*C*T*-3′.

40-46. (canceled)

47. A method for gene profiling, comprising:
a) providing a plurality of single cells;
b) releasing mRNA from each single cell to provide a plurality of individual mRNA samples, wherein each individual mRNA sample is from a single cell;
c) reverse transcribing the individual mRNA samples, performing a template switching reaction to produce cDNA incorporating a barcode sequence, and contacting each individual mRNA sample with a nucleic acid of claim 1 and a nucleic acid of claim 10;
d) pooling and purifying the barcoded cDNA produced from the separate cells;
e) amplifying the barcoded cDNA to generate a cDNA library comprising double-stranded cDNA;
f) purifying the double-stranded cDNA;
g) fragmenting the purified cDNA;
h) purifying the cDNA fragments; and
i) sequencing the cDNA fragments.

48. A method for gene profiling, comprising:
a) providing an isolated population of cells;
b) releasing mRNA from the population of cells to provide one or more mRNA samples;
c) reverse transcribing the one or more mRNA samples, performing a template switching reaction to produce cDNA incorporating a barcode sequence, and contacting each individual mRNA sample with a nucleic acid of claim 1 and a nucleic acid of claim 10;
d) pooling and purifying the barcoded cDNA;
e) amplifying the barcoded cDNA to generate a cDNA library comprising double-stranded cDNA;
f) purifying the double-stranded cDNA;
g) fragmenting the purified cDNA;
h) purifying the cDNA fragments; and
i) sequencing the cDNA fragments.

49. The method of claim 47, further comprising separating a population of cells to provide the plurality of single cells.

50-53. (canceled)

54. The method of claim 47, further comprising contacting the cells with proteinase K.

55-59. (canceled)

60. The method of claim 47, further comprising treating the barcoded cDNA with an exonuclease.

61-70. (canceled)

71. The method of claim 47, wherein the fragmentation of g) utilizes a transposase.

72. The method of claim 71, wherein the fragmentation of g) utilizes a first fragmentation nucleic acid and a second fragmentation nucleic acid, wherein the first fragmentation nucleic acid comprises a barcode sequence.

73. The method of claim 72, wherein the sequence of the first fragmentation nucleic acid is 5′-CAAGCAGAAGACGGCATACGAGAT[i7]GTCTCGTGGGCTCGG-3′, wherein [i7] is a nucleic acid sequence.

74-76. (canceled)

77. The method of claim 72, wherein the barcode sequence of the first fragmentation nucleic acid is different than the barcode sequence of the nucleic acid of claim 10.

78. The method of claim 77, wherein the barcode sequence of the first fragmentation nucleic acid uniquely identifies a predetermined subset of cells.

79. The method of claim 78, wherein the predetermined subset of cells is a subset of cells contained in individual wells of a single capture plate.

80. The method of claim 79, wherein the barcode sequence that uniquely identifies the predetermined subset of cells uniquely identifies the capture plate.

81. The method of claim 77, wherein the barcode sequence of the nucleic acid of claim 10 uniquely identifies the cell within the predetermined subset of cells, which cell comprised the mRNA from which the barcoded cDNA of c) was produced.

82. The method of claim 81, wherein the barcode sequence that uniquely identifies the cell within the predetermined subset of cells uniquely identifies an individual well in a capture plate.

83. The method of claim 82, wherein the combination of the barcode sequence that uniquely identifies the predetermined subset of cells and the barcode sequence that uniquely identifies the cell within a predetermined subset of cells uniquely identifies the capture plate and the individual well which comprised the cell, which cell comprised the mRNA from which the barcoded cDNA of c) was produced.

84-88. (canceled)

89. The method of claim 83, wherein the sequence of the second fragmentation nucleic acid is 5′-AATGATACGGCGACCACCGAGATCTACACTCTTTCCCTACACGACGCTCTTCCG*A*T*C*T*-3′.

90-93. (canceled)

94. The method of claim 47, further comprising assembling a database of the sequences of the sequenced cDNA fragments of j).

95. The method of claim 94, further comprising identifying the UMI sequences of the sequences of the database.

96. The method of claim 95, further comprising discounting duplicate sequences that share a UMI sequence, thereby assembling a set of sequences in which each sequence is associated with a unique UMI.

97-98. (canceled)

99. The method of claim 72, wherein the barcode sequence of the first fragmentation nucleic acid and the barcode sequence of the nucleic acid of claim 10 are used to correlate the sequencing data with the predetermined subset of cells and the individual cell.
